(19) United States
(12) Patent Application Publication
(10) Pub. No.: US 2024/0407946 A1
(43) Pub. Date: Dec. 12, 2024

(54) SUPRACHOROIDAL IMPLANTABLE DEVICES AND METHODS FOR TREATING HYPERTENSION WITHIN AN EYE

(71) Applicant: W. L. Gore & Associates, Inc., Newark, DE (US)

(72) Inventors: Jesse R. McAlister, Newark, DE (US); Kevin M. Savory, West Grove, PA (US); Jeffrey C. Towler, Wilmington, DE (US)

(21) Appl. No.: 18/737,399

(22) Filed: Jun. 7, 2024

Related U.S. Application Data
(60) Provisional application No. 63/471,916, filed on Jun. 8, 2023.
(60) Provisional application No. 63/657,028, filed on Jun. 6, 2024.

(51) Int. Cl. A61F 9/00; A61F 9/007

(52) U.S. Cl. CPC A61F 9/0017; A61F 9/00781; A61F 2210/0057; A61F 2220/0008; A61F 2230/0006; A61F 2230/0008; A61F 2230/0019; A61F 2230/0069; A61F 2250/0023; A61F 2250/0037; A61F 2250/0051

(57) ABSTRACT

Suprachoroidal implantable devices formed of a compliant material have an external surface and an opposing internal surface with varying porosity extending through the compliant material or presenting external and internal surfaces with differing porosities. The compliant material surfaces either facilitate or inhibit tissue ingrowth into the material. The device and methods of using the device reduce pressure in the eye by directing fluid through the compliant material.

### CROSS REFERENCE TO RELATED APPLICATIONS

The present application claims the benefit of U.S. Provisional App. No. 63/471,916 filed Jun. 8, 2023, and U.S. Provisional App. No. 63/657,028 filed Jun. 6, 2024, which are incorporated herein by reference in their entireties for all purposes.

### FIELD

The present disclosure relates generally to apparatuses and methods for treating ocular hypertension. More specifically, the disclosure relates to apparatuses and methods for treating hypertension within an eye via a suprachoroidal implant.

### BACKGROUND

Aqueous humor is a fluid that fills the anterior chamber of the eye and contributes to an intraocular pressure (IOP) or fluid pressure inside the eye. Ocular hypertension is a condition in the eye where the intraocular pressure or fluid pressure inside the eye is elevated. Untreated ocular hypertension can lead to disease, including glaucoma, which can result in a gradual and permanent loss of vision in the afflicted eye. Glaucoma affects more than 3 million people in the United States and is a group of diseases that damages the eye's optic nerve which, if left untreated, can result in vision loss and blindness. Raised IOP is the only modifiable risk factor for glaucoma. Thus, treatment of glaucoma is focused on lowering IOP to reduce the risk of disease progression and vision loss.

Many attempts have been made to treat ocular hypertension, and glaucoma in particular. Such attempts include surgical procedures that involve implantation of drainage devices designed to lower the TOP of the afflicted eye, as well as medicament administration. The goal of these treatments is to improve quality of life and to preserve visual function through a reduction of the IOP. Such treatment procedures vary in their surgical risk, invasiveness and ultimately the effectiveness in lowering IOP.

For example, implantable stents such as the CyPass® stent (Alcon Inc.), the iStent Supra® stent (Glaukos Corp.), the MINIject® stent (iSTAR Medical), and the BioStent® (Iantrek, Inc.) are some of the types of minimally invasive ab-interno suprachoroidal stents that have been used in the past, as summarized in Ianchulev et al. (2024), “Biotissue stent for supraciliary outflow in open-angle glaucoma patients: surgical procedure and first clinical results of an aqueous drainage biostent.” British Journal of Ophthalmology. 2024 Jan. 29; 108 (2): 217-222. doi: 10.1136/bjo-2022-322536. PMID: 36593090; PMCID: PMC10850681 (hereinafter referred to as “Ianchulev et al.”). As explained in Ianchulev et al., the CyPass® and iStent Supra® stents are rigid, non-conforming, non-permeable, and non-hydrophilic, while the MINIject® stent and the BioStent® are porous, hydrophilic, and permeable.

Initial glaucoma treatments are generally focused on enhancing existing aqueous outflow pathways including both the conventional (trabecular) and unconventional (uveoscleral) pathways. As these treatments fail, or the patient's glaucoma becomes un-responsive to treatment, the glaucoma is termed refractive and glaucoma drainage implants are often used.

### SUMMARY

Disclosed herein are suprachoroidal implantable devices and methods of controlling fluid pressure within an eye. Advantages of such devices and methods include controlling tissue ingrowth with respect to the outer surface of the implantable devices. In some examples, the advantages include adjustability of the internal volume of the implantable device in situ such that the internal volume can be controlled without removing the device from the location of implant. In some examples, the devices and methods disclosed herein provide the benefit of minimizing or inhibiting the development of fibrosis in the tissue surrounding the implant, and inhibiting the ingrowth from surrounding tissue through the device, particularly through the internal surface of the device. In various examples, the reservoir or inner volume of the device remains free of tissue ingrowth. In some examples, the external surface allows for tissue ingrowth in order to anchor the device to the location in which it may be implanted and/or supporting the surrounding tissue. The devices can be flexible and cause little or no discomfort to the patient after implant. In various implementations, the devices are capable of exerting outward forces on the external tissue of the eye in a location in which the device is implanted. Such outward forces may help maintain a secondary drainage pathway for aqueous humor to flow from the anterior chamber of the eye. This maintained flow can, in turn, help reduce the intraocular pressure (IOP) within the eye when the primary drainage pathway is obstructed, for example.

According to one example (“Example 1”), a suprachoroidal implantable device includes a body portion formed of a compliant material having an external surface and an internal surface defining an internal reservoir of the body portion with a fixed volume. The external surface has a surface feature that is minimally present on the internal surface when the external and internal surfaces are each viewed at a magnification selected from a range of 50× to 1000×. Or, in some related examples, the external surface is characterized by a surface feature that is minimally present on the internal surface, and further wherein the surface feature is less visible at the internal surface than the external surface, and optionally wherein the surface feature is visible on the external surface at a magnification of 50× and generally not visible on the internal surface at a magnification of 50×, and optionally wherein the surface feature is visible on the external surface at a magnification of 100× and generally not visible on the internal surface at a magnification of 100×, and optionally wherein the surface feature is visible on the external surface at a magnification of 500× and generally not visible on the internal surface at a magnification of 500×, and optionally wherein the surface feature is visible on the external surface at a magnification of 1000× and generally not visible on the internal surface at a magnification of 1000×.

According to another example (“Example 2”) further to Example 1, the surface feature of the external surface includes a plurality of solid portions and a plurality of pore portions. The pore portions include pores of 5 μm to 100 μm in size, the pores being evenly distributed between the solid portions.

According to another example (“Example 3”) further to Example 2, the pore portions are flexible such that the pore are expandable, for example under physiologic conditions.

According to another example (“Example 4”) further to Example 1, the surface feature includes a surface roughness, and the surface roughness of the external surface is greater than the surface roughness of the internal surface.

According to another example (“Example 5”) further to Example 1, the surface feature includes a maximum depth, and the maximum depth of the surface feature at the external surface is greater than the maximum depth of the surface feature at the internal surface.

According to another example (“Example 6”) further to Example 1, the surface feature is defined by a microstructure defined by a plurality of fibrils extending between a plurality of nodes, and further wherein the microstructure is more visible at the external surface than the internal surface.

According to another example (“Example 7”) further to Example 1, the body portion is formed of a material having a microstructure defined by a plurality of fibers.

According to another example (“Example 8”), a suprachoroidal implantable device includes a body portion formed of a compliant material having an external surface and an internal surface defining an internal reservoir of the body portion with a fixed volume. The external surface has a plurality of openings of up to 100 μm in size that are visually unobservable in the internal surface at a magnification, such as a magnification selected from a range of from 50× to 1000×. Or, in some related examples, the external surface has a plurality of openings of up to 100 μm in size that are visually unobservable at 50×, and optionally 100×, and optionally 500×, and optionally 1000×.

According to another example (“Example 9”) further to Example 8, the openings are defined by a plurality of fibrils extending between a plurality of nodes.

According to another example (“Example 10”) further to Example 8, the openings are defined by a plurality of fibers.

According to another example (“Example 11”) further to Example 10, the plurality of fibers include spunbond polymers.

According to another example (“Example 12”) further to any one of Examples 1-11, the internal surface inhibits tissue ingrowth therethrough.

According to another example (“Example 13”), a suprachoroidal implantable device includes a body portion formed of a compliant material having an external surface and an internal surface defining an internal reservoir of the body portion with a fixed volume. The body portion has a variable porosity that transitions from a first porosity located proximate the external surface to a second porosity less than the first porosity and located proximate the internal surface. The first porosity facilitates tissue ingrowth more than the second porosity. Optionally, the first porosity facilitates tissue ingrowth at the external surface and the second porosity inhibits tissue ingrowth through the internal surface.

According to another example (“Example 14”), a suprachoroidal implantable device includes a body portion formed of a compliant material having an external surface and an internal surface defining an internal reservoir of the body portion with a fixed volume. The external surface has a first porosity and the internal surface having a second porosity less than the first porosity. The first porosity facilitates tissue ingrowth more than the second porosity. Optionally, the first porosity facilitates tissue ingrowth at the external surface and the second porosity inhibits tissue ingrowth through the internal surface.

According to another example (“Example 15”) further to Example 13 or 14, the first porosity is defined by a first average pore size, and the second porosity is defined by a second average pore size that is smaller than the first average pore size.

According to another example (“Example 16”) further to any one of Examples 1-15, the external surface is a tissue engagement surface.

According to another example (“Example 17”) further to Example 16, the tissue engagement surface has a porosity extending into the engagement surface at an engagement depth to which an external tissue engages in order to secure or anchor the body portion at a suprachoroidal location in an eye at which the device is implanted.

According to another example (“Example 18”) further to Example 17, the porosity is selected such that the external tissue is observable to engage the engagement surface at the engagement depth after 30 days.

According to another example (“Example 19”) further to Example 17 or 18, the engaging of the tissue engagement surface with the external tissue inhibits migration of the device from the suprachoroidal location.

According to another example (“Example 20”) further to any one of Examples 17-19, the ingrowth of the external tissue on the tissue engagement surface does not significantly inhibit fluid flow through the body portion.

According to another example (“Example 21”) further to any one of Examples 1-20, the body portion is pre-sealed to maintain the fixed volume of the internal reservoir.

According to another example (“Example 22”) further to any one of Examples 1-21, the compliant material includes expanded polytetrafluoroethylene (ePTFE).

According to another example (“Example 23”) further to any one of Examples 1-22, the internal reservoir includes a filler material encapsulated therein.

According to another example (“Example 24”) further to Example 23, the filler material includes ePTFE or hydrogel.

According to another example (“Example 25”) further to Example 23, the filler material includes a medicament, and optionally a medicament selected to pass through the compliant material from the internal surface to the external surface over a period of 30 days.

According to another example (“Example 26”), a suprachoroidal implantable device includes a body portion formed of a compliant material having an external surface and an internal surface defining an internal reservoir of the body portion with an internal volume that is adjustable in situ. The external surface is less uniform than the internal surface when the external and internal surfaces are each viewed at a magnification, such as a magnification selected from a range of from 50× to 1000×. Or, in some related examples, when the external and internal surfaces are each viewed at a magnification of 50×, and optionally 100×, and optionally 500×, and optionally 1000×, the external surface can be observed as being less uniform than the internal surface.

According to another example (“Example 27”), a suprachoroidal implantable device includes a body portion formed of a compliant material having an external surface and an internal surface defining an internal reservoir of the body portion with an internal volume that is adjustable in situ. The body portion has a variable porosity that transitions from a first porosity located proximate the external surface to a second porosity less than the first porosity and located proximate the internal surface. The first porosity facilitates tissue ingrowth at the external surface and the second porosity inhibits tissue ingrowth through the internal surface.

According to another example (“Example 28”), a suprachoroidal implantable device includes a body portion formed of a compliant material having an external surface and an internal surface defining an internal reservoir of the body portion with an internal volume that is adjustable in situ. The external surface has a first porosity and the internal surface having a second porosity less than the first porosity. The first porosity facilitates tissue ingrowth at the external surface and the second porosity inhibits tissue ingrowth through the internal surface.

According to another example (“Example 29”) further to Example 27 or 28, the first porosity is defined by a first average pore size, and the second porosity is defined by a second average pore size that is smaller than the first average pore size.

According to another example (“Example 30”) further to any one of Examples 26-29, the external surface is a tissue engagement surface.

According to another example (“Example 31”) further to Example 30, the tissue engagement surface has a porosity extending into the engagement surface at an engagement depth to which an external tissue engages in order to secure or anchor the body portion at a suprachoroidal location in an eye at which the device is implanted.

According to another example (“Example 32”) further to Example 31, the porosity is selected such that the external tissue is observable to engage the engagement surface at the engagement depth after 30 days.

According to another example (“Example 33”) further to Example 31 or 32, the engaging of the tissue engagement surface with the external tissue inhibits migration of the device from the suprachoroidal location under physiologic conditions.

According to another example (“Example 34”) further to any one of Examples 31-33, the ingrowth of the external tissue on the tissue engagement surface does not significantly inhibit fluid flow through the body portion.

According to another example (“Example 35”) further to any one of Examples 26-34, the body portion is pre-sealed prior to implanting of the device.

According to another example (“Example 36”) further to any one of Examples 26-35, the body portion comprises ePTFE.

According to another example (“Example 37”) further to any one of Examples 26-36, the body portion has a maximum internal capacity and is partially pre-filled to less than the internal maximum capacity to define the internal volume.

According to another example (“Example 38”) further to Example 37, the body portion is partially pre-filled with a filler material to fill at least 10% of a maximum capacity of the internal volume of the internal reservoir.

According to another example (“Example 39”) further to Example 38, the filler material includes ePTFE, hydrogel, or combinations thereof.

According to another example (“Example 40”) further to Example 38, the filler material includes a medicament, and optionally a medicament selected to, or that otherwise passes through the compliant material from the internal surface to the external surface over a period of 30 days.

According to another example (“Example 41”) further to any one of Examples 26-40, the device further includes a sealable conduit having a first end and a second end fluidly coupled with the internal reservoir to facilitate in situ adjustment to the internal volume of the internal reservoir.

According to another example (“Example 42”) further to Example 41, the body portion and the sealable conduit comprise ePTFE.

According to another example (“Example 43”) further to Example 41 or 42, the first end of the sealable conduit is disposable in a subconjunctival location of an eye, and the first end is sealable after the in situ adjustment of the internal volume.

According to another example (“Example 44”) further to Example 43, the subconjunctival location is located between a conjunctival tissue and a scleral tissue of the eye.

According to another example (“Example 45”) further to Example 41, the first end of the sealable conduit is disposable in an anterior chamber (AC) of an eye.

According to another example (“Example 46”) further to Example 45, the first end is sealable after the in situ adjustment of the internal volume.

According to another example (“Example 47”) further to Example 45, the first end of the sealable conduit remains open to facilitate the in situ adjustment of the internal volume of the internal reservoir.

According to another example (“Example 48”), a suprachoroidal implantable device includes a body portion having a closed end and an open end fluidly couplable with an anterior chamber (AC) of an eye. The body portion includes a first surface and a second surface opposite the first surface, and that is less uniform than the first surface when the first and second surfaces are each viewed at a magnification, for example a magnification selected from a range of from 50× to 1000×. Or, in some related examples, when the first and second surfaces are each viewed at a magnification of 50×, and optionally 100×, and optionally 500×, and optionally 1000×, the second surface can be observed as being less uniform than the first surface.

According to another example (“Example 49”), a suprachoroidal implantable device includes a body portion having a closed end and an open end fluidly couplable with an anterior chamber (AC) of an eye. The body portion includes a first surface and a second surface, the body portion having a variable porosity that transitions from a first porosity located proximate the first surface to a second porosity less than the first porosity and located proximate the second surface. The first porosity facilitates tissue ingrowth at the first surface and the second porosity inhibits tissue ingrowth through the second surface.

According to another example (“Example 50”), a suprachoroidal implantable device includes a body portion having a closed end and an open end fluidly couplable with an anterior chamber (AC) of an eye. The body portion includes a first surface and a second surface, the first surface having a first porosity, and the second surface having a second porosity less than the first porosity. The first porosity facilitates tissue ingrowth at the first surface and the second porosity inhibits tissue ingrowth through the second surface.

According to another example (“Example 51”) further to Example 49 or 50, the first porosity is defined by a first average pore size, and the second porosity is defined by a second average pore size that is smaller than the first average pore size.

According to another example (“Example 52”) further to any one of Examples 48-51, the external surface is a tissue engagement surface.

According to another example (“Example 53”) further to Example 52, the tissue engagement surface has a porosity at a depth with which an external tissue engages in order to secure or anchor the body portion at a suprachoroidal location in an eye at which the device is implanted.

According to another example (“Example 54”) further to Example 53, the engaging of the tissue engagement surface with the external tissue is observable after 30 days.

According to another example (“Example 55”) further to Example 53 or 54, the engaging of the tissue engagement surface with the external tissue inhibits migration of the device from the suprachoroidal location.

According to another example (“Example 56”) further to any of Examples 53-55, the ingrowth of the external tissue on the tissue engagement surface does not significantly inhibit fluid flow through the body portion.

According to another example (“Example 57”) further to any of Examples 48-56, the body portion comprises ePTFE.

According to another example (“Example 58”) further to any of Examples 48-57, the body portion is substantially hollow.

According to another example (“Example 59”) further to any of Examples 48-58, the body portion facilitates fluid communication therethrough from the second surface to the first surface in response to a fluid pressure applied from the AC.

According to another example (“Example 60”) further to Example 59, fluid from the AC is dispersible via the first surface to an external location within the eye.

According to another example (“Example 61”) further to Example 60, the external location is between a conjunctival tissue and a scleral tissue of the eye.

According to another example (“Example 62”) further to any of Examples 48-61, the body portion is a tubular construct having a first end and a second end, wherein the first end is closed to form the closed end of the body portion and the second end is retained in an open configuration to form the open end of the body portion.

According to another example (“Example 63”) further to Example 62, the first end is closed by pinching the first end of the tubular construct.

According to another example (“Example 64”) further to any of Examples 48-63, the body portion defines an internal region and an external region configured to directly contact an external tissue within an eye, the second surface covers an entirety of the internal region and a portion of the external region, and the first surface covers the remaining portion of the external region.

According to another example (“Example 65”) further to any one of Examples 48-64, the body portion has a substantially circular cross-section.

According to another example (“Example 66”) further to any one of Examples 48-64, the body portion has a substantially ovular or rounded rectangular cross-section.

According to another example (“Example 67”) further to Example 66, in an absence of external forces, the body portion assumes a first configuration that has a first height and a first width, and in response to the external forces being applied to the body portion, the body portion assumes a second configuration that has a second height and a second width, wherein the second height is less than the first height, and the second width is greater than the first width.

According to another example (“Example 68”) further to Example 67, the body portion reversibly transitions between the first configuration and the second configuration in the presence or absence of the external forces being applied to the body portion.

According to another example (“Example 69”) further to any of Examples 48-67, the body portion includes: an external microporous layer, and an internal elastic support structure disposed within the external microporous layer and defining an internal space. The external microporous layer defines both the first surface and the second surface, and further wherein the internal elastic support structure has a third porosity.

According to another example (“Example 70”) further to Example 69, the third porosity is greater than the first porosity and the second porosity to facilitate fluid communication between the internal space and the second surface.

According to another example (“Example 71”), a method of controlling fluid pressure within an eye is disclosed. The method includes providing a suprachoroidal implantable device having a body portion formed of a compliant material and having an external surface and an opposing internal surface defining an internal reservoir with a fixed volume and disposing the device in a subconjunctival location of the eye. The external surface is less uniform than the internal surface when the external and internal surfaces are each viewed at a magnification, for example a magnification selected from a range of from 50× to 1000×. Or, in some related examples, when the external and internal surfaces are each viewed at a magnification of 50×, and optionally 100×, and optionally 500×, and optionally 1000×, the external surface can be observed as being less uniform than the internal surface. The method also includes controlling fluid pressure in the eye by directing a fluid through the compliant material.

According to another example (“Example 72”), another method of controlling fluid pressure within an eye is disclosed. The method includes: providing a suprachoroidal implantable device having a body portion formed of a compliant material and having an external surface and an opposing internal surface defining an internal reservoir with a fixed volume, the body portion having a variable porosity that transitions from a first porosity located proximate the external surface to a second porosity less than the first porosity and located proximate the internal surface; disposing the device in a subconjunctival location of the eye; and controlling fluid pressure in the eye by directing a fluid through the compliant material. The first porosity facilitates tissue ingrowth at the external surface and the second porosity inhibits, and optionally prevents, tissue ingrowth through the internal surface.

According to another example (“Example 73”), another method of controlling fluid pressure within an eye is disclosed. The method includes: providing a suprachoroidal implantable device having a body portion formed of a compliant material and having an external surface and an opposing internal surface defining an internal reservoir with a fixed volume, the external surface having a first porosity, the internal surface having a second porosity less than the first porosity; disposing the device in a subconjunctival location of the eye; and controlling fluid pressure in the eye by directing a fluid through the compliant material. The first porosity facilitates tissue ingrowth at the external surface and the second porosity inhibits, and optionally prevents, tissue ingrowth through the internal surface.

According to another example (“Example 74”), another method of controlling fluid pressure within an eye is disclosed. The method includes: providing a suprachoroidal implantable device having a body portion formed of a compliant material and having an external surface and an opposing internal surface defining an internal reservoir with an internal volume; disposing the device in a subconjunctival location of the eye, wherein the external surface is less uniform than the internal surface when the external and internal surfaces are each viewed at a magnification selected from a range of 50× to 1000× (or, in some related examples, when the external and internal surfaces are each viewed at a magnification of 50×, and optionally 100×, and optionally 500×, and optionally 1000×, the external surface can be observed as being less uniform than the internal surface); adjusting in situ the internal volume of the internal reservoir; and controlling fluid pressure in the eye by directing a fluid through the compliant material.

According to another example (“Example 75”), another method of controlling fluid pressure within an eye is disclosed. The method includes: providing a suprachoroidal implantable device having a body portion formed of a compliant material and having an external surface and an opposing internal surface defining an internal reservoir with an internal volume, the body portion having a variable porosity that transitions from a first porosity located proximate the external surface to a second porosity less than the first porosity and located proximate the internal surface; disposing the device in a subconjunctival location of the eye, wherein the first porosity facilitates tissue ingrowth at the external surface and the second porosity inhibits tissue ingrowth through the internal surface; adjusting in situ the internal volume of the internal reservoir; and controlling fluid pressure in the eye by directing a fluid through the compliant material.

According to another example (“Example 76”), another method of controlling fluid pressure within an eye is disclosed. The method includes: providing a suprachoroidal implantable device having a body portion formed of a compliant material and having an external surface and an opposing internal surface defining an internal reservoir with an internal volume, the external surface having a first porosity, the internal surface having a second porosity less than the first porosity; disposing the device in a subconjunctival location of the eye, wherein the first porosity facilitates tissue ingrowth at the external surface and the second porosity inhibits tissue ingrowth through the internal surface; adjusting in situ the internal volume of the internal reservoir; and controlling fluid pressure in the eye by directing a fluid through the compliant material.

According to another example (“Example 77”) further to any one of Examples 74-76, the method further includes: providing a sealable conduit having a first end and a second end; fluidly coupling the second end of the sealable conduit with the internal reservoir of the device, wherein the sealable conduit facilitates the adjusting in situ of the internal volume of the internal reservoir; and disposing the first end of the sealable conduit between a conjunctival tissue and a scleral tissue of the eye or in an anterior chamber (AC) of the eye.

According to another example (“Example 78”) further to Example 77, the disposing the first end of the sealable conduit between a conjunctival tissue and a scleral tissue of the eye or in an anterior chamber (AC) of the eye further includes: sealing the first end of the conduit to inhibit fluid communication therethrough.

According to another example (“Example 79”) further to Example 78, the disposing the first end of the sealable conduit in an AC of the eye further includes: fluidly coupling the AC and the first end of the conduit to facilitate fluid communication therethrough from the internal surface to the external surface in response to a fluid pressure applied from the AC.

According to another example (“Example 80”), another method of controlling fluid pressure within an eye is disclosed. The method includes: providing a suprachoroidal implantable device having a body portion having a closed end and an open end, the body portion comprising a first surface and an opposing second surface; disposing the device in a subconjunctival location of the eye such that the open end of the device is fluidly coupled with an anterior chamber (AC) of the eye; and controlling fluid pressure in the eye by directing a fluid through the first and second surfaces. The first surface is less uniform than the second surface when the first and second surfaces are each viewed at a magnification selected from a range of 50× to 1000×. Or, in some related examples, when the first and second surfaces are each viewed at a magnification of 50×, and optionally 100×, and optionally 500×, and optionally 1000×, the first surface can be observed as being less uniform than the second surface.

According to another example (“Example 81”), another method of controlling fluid pressure within an eye is disclosed. The method includes: providing a suprachoroidal implantable device having a body portion having a closed end and an open end, the body portion having a first surface and an opposing second surface, the body portion having a variable porosity that transitions from a first porosity located proximate the first surface to a second porosity less than the first porosity and located proximate the second surface; disposing the device in a subconjunctival location of the eye such that the open end of the device is fluidly coupled with an anterior chamber (AC) of the eye; and controlling fluid pressure in the eye by directing a fluid through the first and second porosities. The first porosity facilitates tissue ingrowth at the first surface and the second porosity inhibits tissue ingrowth through the second surface.

According to another example (“Example 82”), another method of controlling fluid pressure within an eye is disclosed. The method includes: providing a suprachoroidal implantable device having a body portion having a closed end and an open end, the body portion having a first surface and an opposing second surface, the first surface having a first porosity, and the second surface having a second porosity less than the first porosity; disposing the device in a subconjunctival location of the eye such that the open end of the device is fluidly coupled with an anterior chamber (AC) of the eye; and controlling fluid pressure in the eye by directing a fluid through the first and second porosities. The first porosity facilitates tissue ingrowth at the first surface and the second porosity inhibits, tissue ingrowth through the second surface.

According to another example (“Example 83”), a method of manufacturing a suprachoroidal implantable device is disclosed. The method includes: providing a compliant material; forming a body portion using the compliant material such that the body portion comprises an external surface and an internal surface, the external surface being less uniform than the internal surface at a magnification of from about 50× to about 1000× magnification to facilitate tissue ingrowth (or, in some related examples, when the external and internal surfaces are each viewed at a magnification of 50×, and optionally 100×, and optionally 500×, and optionally 1000×, the external surface can be observed as being less uniform than the internal surface); and providing the body portion with a filler material therein to define an internal reservoir with a fixed volume.

According to another example (“Example 84”), another method of manufacturing a suprachoroidal implantable device is disclosed. The method includes: providing a compliant material; forming a body portion using the compliant material such that the body portion comprises an external surface and an internal surface, the body portion having a variable porosity that transitions from a first porosity located proximate the external surface to a second porosity less than the first porosity and located proximate the internal surface; and providing the body portion with a filler material therein to define an internal reservoir with a fixed volume.

According to another example (“Example 85”), another method of manufacturing a suprachoroidal implantable device is disclosed. The method includes: providing a compliant material with a first porosity on a first surface and a second porosity that is less than the first porosity on a second surface; forming a body portion using the compliant material such that the body portion comprises an external surface and an internal surface, the external surface having the first porosity to facilitate tissue ingrowth at the external surface, and the internal surface having the second porosity to inhibit tissue ingrowth through the internal surface; and providing the body portion with a filler material therein to define an internal reservoir with a fixed volume.

According to another example (“Example 86”), another method of manufacturing a suprachoroidal implantable device is disclosed. The method includes: providing a compliant material; and forming a body portion using the compliant material such that the body portion comprises an external surface and an internal surface, the external surface being less uniform than the internal surface at a magnification of from about 50× to about 1000× magnification to facilitate tissue ingrowth (or, in some related examples, when the external and internal surfaces are each viewed at a magnification of 50×, and optionally 100×, and optionally 500×, and optionally 1000×, the external surface can be observed as being less uniform than the internal surface), wherein the internal surface defines an internal reservoir with an internal volume that is adjustable in situ.

According to another example (“Example 87”), another method of manufacturing a suprachoroidal implantable device is disclosed. The method includes: providing a compliant material with a first porosity on a first surface and a second porosity that is less than the first porosity on a second surface; and forming a body portion using the compliant material such that the body portion comprises an external surface and an internal surface, the body portion having a variable porosity that transitions from a first porosity located proximate the external surface to a second porosity less than the first porosity and located proximate the internal surface, wherein the internal surface defines an internal reservoir with an internal volume that is adjustable in situ.

According to another example (“Example 88”), another method of manufacturing a suprachoroidal implantable device is disclosed. The method includes: providing a compliant material with a first porosity on a first surface and a second porosity that is less than the first porosity on a second surface; and forming a body portion using the compliant material such that the body portion comprises an external surface and an internal surface, the external surface having the first porosity that facilitates tissue ingrowth at the external surface, and the internal surface having the second porosity that inhibits tissue ingrowth through the internal surface, wherein the internal surface defines an internal reservoir with an internal volume that is adjustable in situ.

According to another example (“Example 89”) further to any one of Examples 86-88, the method further includes: providing a sealable conduit having a first end and a second end fluidly coupled with the internal reservoir to facilitate in situ adjustment to the internal volume of the internal reservoir.

According to another example (“Example 90”), another method of manufacturing a suprachoroidal implantable device is disclosed. The method includes: wrapping around a mandrel a compliant material; and applying heat treatment to the compliant material to form a body portion having a closed end and an open end configured to be fluidly coupled with an anterior chamber (AC) of an eye, the body portion comprising a first surface and a second surface, the first surface being less uniform than the second surface at a magnification of from about 50× to about 1000× magnification to facilitate tissue ingrowth. Or, in some related examples, when the first and second surfaces are each viewed at a magnification of 50×, and optionally 100×, and optionally 500×, and optionally 1000×, the first surface can be observed as being less uniform than the second surface.

According to another example (“Example 91”), another method of manufacturing a suprachoroidal implantable device is disclosed. The method includes: wrapping around a mandrel a compliant material; and applying heat treatment to the compliant material to form a body portion having a closed end and an open end fluidly couplable with an anterior chamber (AC) of an eye, the body portion comprising a first surface and a second surface, the body portion comprising a first surface and a second surface, the body portion having a variable porosity that transitions from a first porosity located proximate the first surface to a second porosity less than the first porosity and located proximate the second surface, the first surface having the first porosity to facilitate tissue ingrowth at the first surface of the body portion, and the second surface having the second porosity to inhibit tissue ingrowth through the second surface of the body portion.

According to another example (“Example 92”), another method of manufacturing a suprachoroidal implantable device is disclosed. The method includes: wrapping around a mandrel a compliant material with a first porosity on a first surface and a second porosity that is less than the first porosity on a second surface; and applying heat treatment to the compliant material to form a body portion having a closed end and an open end fluidly couplable with an anterior chamber (AC) of an eye, the body portion comprising a first surface and a second surface, the first surface having the first porosity to facilitate tissue ingrowth at the first surface of the body portion, and the second surface having the second porosity to inhibit tissue ingrowth through the second surface of the body portion.

According to another example (“Example 93”) further to any one of Examples 90-92, the method further includes: prior to wrapping a compliant material around a mandrel, wrapping around the mandrel a secondary compliant material. Wrapping a compliant material around a mandrel includes wrapping the compliant material around the secondary compliant material. Furthermore, applying the heat treatment to the compliant material includes: applying the heat treatment to the compliant material to form an external microporous layer, wherein the external microporous layer defines both the first surface and the second surface of the body portion; and applying the heat treatment to the secondary compliant material to form an internal elastic support structure disposed within the external microporous layer and defining an internal space, wherein the internal elastic support structure has a third porosity greater than the first porosity and the second porosity to facilitate fluid communication between the internal space and the second surface.

According to another example (“Example 94”), a method of reducing a fluid pressure of a fluid within an eye includes: disposing a suprachoroidal implantable device in a subconjunctival location of the eye, the device having a body portion formed of a compliant material with an external surface and an opposing internal surface defining an internal reservoir of the device having a fixed volume, wherein the external surface is less uniform than the internal surface when both the external and internal surfaces are each viewed at a magnification selected from a range of 50× to 1000×; and reducing the fluid pressure by conveying the fluid though the compliant material from a high-pressure location of the eye to a low-pressure location.

According to another example (“Example 95”), a method of reducing a fluid pressure of a fluid within an eye includes: disposing a suprachoroidal implantable device in a subconjunctival location of the eye, the device having a body portion formed of a compliant material with an external surface and an opposing internal surface defining an internal reservoir of the device having a fixed volume, the compliant material having a variable porosity that transitions from a first porosity located proximate the external surface to a second porosity less than the first porosity and located proximate the internal surface, wherein the first porosity facilitates tissue ingrowth into the external surface and the second porosity inhibits tissue ingrowth into the internal surface; and reducing the fluid pressure by conveying the fluid though the compliant material from a high-pressure location of the eye to a low-pressure location.

According to another example (“Example 96”), a method of reducing a fluid pressure of a fluid within an eye includes: disposing a suprachoroidal implantable device in a subconjunctival location of the eye, the device having a body portion formed of a compliant material with an external surface and an opposing internal surface defining an internal reservoir of the device having a fixed volume, the external surface having a first porosity and the internal surface having a second porosity less than the first porosity, wherein the first porosity facilitates tissue ingrowth into the external surface and the second porosity inhibits tissue ingrowth into the internal surface; and reducing the fluid pressure by conveying the fluid though the compliant material from a high-pressure location of the eye to a low-pressure location.

According to another example (“Example 97”), a method of reducing a fluid pressure of a fluid within an eye includes: disposing a suprachoroidal implantable device in a subconjunctival location of the eye, the device having a body portion formed of a compliant material with an external surface and an opposing internal surface defining an internal reservoir of the device having an internal volume, wherein the external surface is less uniform than the internal surface when both the external and internal surfaces are each viewed at a magnification selected from a range of 50× to 1000×; adjusting in situ the internal volume of the internal reservoir; and reducing the fluid pressure by conveying the fluid though the compliant material from a high-pressure location of the eye to a low-pressure location.

According to another example (“Example 98”), a method of reducing a fluid pressure of a fluid within an eye includes: disposing a suprachoroidal implantable device in a subconjunctival location of the eye, the device having a body portion formed of a compliant material with an external surface and an opposing internal surface defining an internal reservoir of the device having an internal volume, the compliant material having a variable porosity that transitions from a first porosity located proximate the external surface to a second porosity less than the first porosity and located proximate the internal surface, wherein the first porosity facilitates tissue ingrowth into the external surface and the second porosity inhibits tissue ingrowth into the internal surface; adjusting in situ the internal volume of the internal reservoir; and reducing the fluid pressure by conveying the fluid though the compliant material from a high-pressure location of the eye to a low-pressure location.

According to another example (“Example 99”), a method of reducing a fluid pressure of a fluid within an eye includes: disposing a suprachoroidal implantable device in a subconjunctival location of the eye, the device having a body portion formed of a compliant material with an external surface and an opposing internal surface defining an internal reservoir with an internal volume, the external surface having a first porosity and the internal surface having a second porosity less than the first porosity, wherein the first porosity facilitates tissue ingrowth into the external surface and the second porosity inhibits tissue ingrowth into the internal surface; adjusting in situ the internal volume of the internal reservoir; and reducing the fluid pressure by conveying the fluid though the compliant material from a high-pressure location of the eye to a low-pressure location.

According to another example (“Example 100”) further to any one of Examples 97-99, the method further includes: disposing a first end of a sealable conduit between a conjunctival tissue and at least one of scleral tissue of the eye and an anterior chamber (AC) of the eye, the sealable conduit having a second end opposing the first end; and fluidly coupling the second end of the sealable conduit with the internal reservoir of the device, wherein the sealable conduit facilitates the adjusting in situ of the internal volume of the internal reservoir.

According to another example (“Example 101”) further to Example 100, the disposing of the first end of the sealable conduit further includes sealing the first end of the conduit to inhibit fluid communication therethrough.

According to another example (“Example 102”) further to Example 101, the disposing the first end of the sealable conduit in an AC of the eye further includes fluidly coupling the AC and the first end of the conduit to facilitate fluid communication therethrough from the internal surface to the external surface in response to a fluid pressure applied from the AC.

According to another example (“Example 103”), a method of reducing a fluid pressure of a fluid within an eye includes: disposing a suprachoroidal implantable device in a subconjunctival location of the eye, the device having a body portion defining a closed end and an open end, the body portion further comprising a first surface and an opposing second surface, wherein the open end is fluidly coupled with an anterior chamber (AC) of the eye, and wherein the first surface is less uniform than the second surface when the first and second surfaces are each viewed at a magnification selected from a range of 50× to 1000×; and reducing the fluid pressure by conveying the fluid though the first and second surfaces from a high-pressure location of the eye to a low-pressure location.

According to another example (“Example 104”), a method of reducing a fluid pressure of a fluid within an eye includes: disposing a suprachoroidal implantable device in a subconjunctival location of the eye, the device having a body portion defining a closed end and an open end, the body portion further comprising a first surface and an opposing second surface, the body portion having a variable porosity that transitions from a first porosity located proximate the first surface to a second porosity less than the first porosity and located proximate the second surface, wherein the open end of the device is fluidly coupled with an anterior chamber (AC) of the eye, and wherein the first porosity facilitates tissue ingrowth into the first surface and the second porosity inhibits tissue ingrowth into the second surface; and reducing the fluid pressure by conveying the fluid though the first and second surfaces from a high-pressure location of the eye to a low-pressure location.

According to another example (“Example 105”), a method of reducing a fluid pressure of a fluid within an eye includes: disposing a suprachoroidal implantable device in a subconjunctival location of the eye, the device having a body portion defining a closed end and an open end, the body portion further comprising a first surface and an opposing second surface, the first surface having a first porosity and the second surface having a second porosity less than the first porosity, wherein the open end of the device is fluidly coupled with an anterior chamber (AC) of the eye, and wherein the first porosity facilitates tissue ingrowth into the first surface and the second porosity inhibits tissue ingrowth into the second surface; and reducing the fluid pressure by conveying the fluid though the first and second surfaces from a high-pressure location of the eye to a low-pressure location.

According to another example (“Example 106”), a suprachoroidal implantable device may include a body having an external surface and an internal surface, the body being formed of a compliant material having a thickness defining an ingrowth portion that facilitates tissue ingrowth and a boundary portion that inhibits tissue ingrowth, the ingrowth portion corresponding to the external surface and the boundary portion defining an internal reservoir within the body, the ingrowth portion being characterized by visible microporous surface features at a first magnification and the boundary portion being characterized by an absence of visible microporous surface features at the first magnification.

According to another example (“Example 107”) further to Example 106, the first magnification is 50×, optionally 100×, optionally 500×, or optionally 1000×.

According to another example (“Example 108”) further to Example 106 or 107, the microporous surface features are defined by a node-and-fibril microstructure or a fiber microstructure.

According to another example (“Example 109”) further to any one of Examples 106-108, the internal reservoir has a fixed internal volume.

According to another example (“Example 110”), a suprachoroidal implantable device includes a body having a closed end and an open end fluidly couplable with an anterior chamber (AC) of an eye, a body having an external surface and an internal surface, the body being formed of a material having a thickness defining an ingrowth portion that facilitates tissue ingrowth and a boundary portion that inhibits tissue ingrowth, the ingrowth portion defining at least a portion of the external surface and the boundary portion defining at least a portion of the internal surface of the body, such that the portion of the external surface defined by the ingrowth portion appears less uniform when inspected at a first magnification than the portion of the internal surface defined by the boundary portion.

According to another example (“Example 111”) further to Example 110, the boundary portion also defines a portion of the external surface of the body.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the embodiments of the disclosure and are incorporated in and constitute a part of this specification, illustrate examples, and together with the description serve to explain the principles of the disclosure.

FIG. 1 is a schematic diagram of a cross-sectional diagrams of an eye with a treatment device implanted to facilitate treatment of hypertension within an eye according to embodiments disclosed herein;

FIG. 2A is a schematic diagram of a cross-sectional view of a suprachoroidal implantable device according to embodiments disclosed herein;

FIG. 2B is a schematic diagram of a cross-sectional view of a suprachoroidal implantable device according to embodiments disclosed herein;

FIG. 2C is a cross-sectional diagram of an eye with the implantable device of FIG. 2A or 2B implanted to facilitate lowering an intraocular pressure (IOP) or fluid pressure inside the eye according to embodiments disclosed herein;

FIG. 3A is a schematic diagram of a cross-sectional view of a suprachoroidal implantable device according to embodiments disclosed herein;

FIG. 3B is a schematic diagram of a cross-sectional view of a suprachoroidal implantable device according to embodiments disclosed herein;

FIG. 3C is a cross-sectional diagram of an eye with the implantable device of FIG. 3A or 3B implanted to facilitate lowering the IOP inside the eye according to embodiments disclosed herein;

FIG. 3D is a cross-sectional diagram of an eye with the implantable device of FIG. 3A or 3B implanted to facilitate lowering the IOP inside the eye according to embodiments disclosed herein;

FIG. 3E is a cross-sectional diagram of an eye with the implantable device of FIG. 3A or 3B implanted to facilitate lowering the IOP inside the eye according to embodiments disclosed herein;

FIG. 4A is a schematic diagram of a cross-sectional view of a suprachoroidal implantable device according to embodiments disclosed herein;

FIG. 4B is a schematic diagram of a front side of the implantable device of FIG. 4A as seen from the direction indicated by the line 4B-4B, according to embodiments disclosed herein;

FIG. 4C is a schematic diagram of a cross-sectional view of the implantable device as cut across line 4C-4C shown in FIG. 4A, according to embodiments disclosed herein;

FIG. 4D is a cross-sectional diagram of an eye with the implantable device of FIG. 4A, 4B, or 4C implanted to facilitate lowering the IOP inside the eye according to embodiments disclosed herein;

FIG. 4E is a schematic diagram of a cross-sectional view of a suprachoroidal implantable device according to embodiments disclosed herein;

FIG. 4F is a schematic diagram of a cross-sectional view of a suprachoroidal implantable device according to embodiments disclosed herein;

FIG. 5A is a schematic diagram of a cross-sectional view of a suprachoroidal implantable device according to embodiments disclosed herein;

FIG. 5B is a schematic diagram of a front side of the implantable device of FIG. 5A as seen from the direction indicated by the line 5B-5B, according to embodiments disclosed herein;

FIG. 5C is a schematic diagram of a cross-sectional view of the implantable device as cut across line 50-5C shown in FIG. 5A, according to embodiments disclosed herein;

FIG. 5D is a cross-sectional diagram of an eye with the implantable device of FIG. 5A, 5B, or 5C implanted to facilitate lowering the IOP inside the eye according to embodiments disclosed herein;

FIG. 6 is an SEM (scanning electron microscopy) image of a cross-sectional view of a body portion of the implantable device according to embodiments disclosed herein, shown to scale as included in the image;

FIGS. 7A and 7B are SEM images with 50× magnification of the surfaces of a body portion of the implantable device according to embodiments disclosed herein, shown to scale as included in the images;

FIGS. 8A and 8B are SEM images with 100× magnification of the surfaces of a body portion of the implantable device according to embodiments disclosed herein, shown to scale as included in the images;

FIGS. 9A and 9B are SEM images with 500× magnification of the surfaces of a body portion of the implantable device according to embodiments disclosed herein, shown to scale as included in the images;

FIGS. 10A and 10B are SEM images with 1000× magnification of the surfaces of a body portion of the implantable device according to embodiments disclosed herein, shown to scale as included in the images;

FIG. 11 is an SEM image of a fiber network microstructure in an external surface of a body portion of the implantable device according to embodiments disclosed herein;

FIG. 12A is a color-coded surface profilometry image of the first or external surface of the body portion according to embodiments disclosed herein, shown to scale as included in the image;

FIG. 12B is a monochromatic surface profilometry image of the surface shown in FIG. 12A, shown to scale as included in the image;

FIG. 12C is an angled color-coded surface profilometry image of the surface shown in FIG. 12A, shown to scale as included in the image;

FIG. 12D is a software legend window showing the characteristics of the surface shown in FIG. 12A;

FIGS. 13A and 13B are cross-sectional views of an implantable device having an ovoid shape, before and after applying directional forces as shown, according to embodiments disclosed herein;

FIGS. 14A and 14B are cross-sectional views of an implantable device having a rounded rectangular shape, before and after applying directional forces as shown, according to embodiments disclosed herein;

FIGS. 15A and 15B are cross-sectional views of a filled implantable device having an ovoid shape, before and after applying directional forces as shown, according to embodiments disclosed herein;

FIGS. 16A and 16B are cross-sectional views of a filled implantable device having a rounded rectangular shape, before and after applying directional forces as shown, according to embodiments disclosed herein; and

FIG. 17 is a cross-sectional diagram of an eye with the implantable device of FIG. 15A, 15B, 16A, or 16B implanted to facilitate lowering the IOP inside the eye according to embodiments disclosed herein.

It should be understood that some of the drawings and replicas of the photographs may not necessarily be shown to scale. In certain instances, details that are not necessary for an understanding of the disclosure or that render other details difficult to perceive may have been omitted. It should be understood, of course, that the disclosure is not necessarily limited to the particular examples or embodiments illustrated or depicted herein.

### DETAILED DESCRIPTION

### Definitions and Terminology

This disclosure is not meant to be read in a restrictive manner. For example, the terminology used in the application should be read broadly in the context of the meaning those in the field would attribute such terminology. Persons skilled in the art will readily appreciate that the various embodiments of the inventive concepts provided in the present disclosure can be realized by any number of methods and apparatuses configured to perform the intended functions. It should also be noted that the accompanying figures referred to herein are not necessarily drawn to scale, but may be exaggerated to illustrate various aspects of the present disclosure, and in that regard, the figures should not be construed as limiting. Some figures do, however, represent anatomy and the positioning of embodiments relative to that anatomy and such representations should be understood to be scaled and positioned accurately, with some deviation permitted as the anatomical structures depicted will vary in size and position from person to person.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

As used herein, the terms or phrases “prevent tissue ingrowth” and “substantially inhibit tissue ingrowth” or “substantially inhibit tissue ingrowth” are meant to be inclusive of not only complete prevention, but also minor, inconsequential ingrowth that does not substantially impact device performance in the context described.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The term “fibril” as used herein describes an elongated piece of material such as a polymer, where the length and width are substantially different from each other. For example, a fibril may resemble a piece of string or fiber, where the width (or thickness) is much shorter or smaller than the length. In some examples, a fibril may be smaller (microscopic) in width or thickness than a piece of fiber.

The term “node” as used herein describes a connection point of at least two fibrils, where the connection may be defined as a location where the two fibrils come into contact with each other, permanently or temporarily. In some examples, a node may also be used to describe a larger volume of polymer than a fibril and where a fibril originates or terminates with no clear continuation of the same fibril through the node. In some examples, a node has a greater width but a smaller length than the fibril.

As used herein, “nodes” and “fibrils” may be used to describe objects that are usually, but not necessarily, connected or interconnected, and have a microscopic size, for example. A “microscopic” object may be defined as an object with at least one dimension (width, length, or height) that is substantially small such that the object or the detail of the object is not visible to the naked eye or difficult, if not impossible, to observe without the aid of a microscope (including but not limited to a scanning electron microscope or SEM, for example) or any suitable type of magnification device.

### DESCRIPTION OF VARIOUS EMBODIMENTS

The present disclosure relates to systems, devices, and methods for lowering an intraocular pressure (IOP) or fluid pressure within an eye of a patient to treat glaucoma. In various embodiments, the treatment device or method is configured to treat, for example, ocular hypertension and/or glaucoma, by causing the IOP to decrease from undesirably high levels that may lead to a gradual and sometimes permanent loss of vision in the afflicted eye. In various embodiments, the suprachoroidal implantable devices according to the instant disclosure are configured to facilitate tissue ingrowth at an external surface, or tissue ingrowth portion of the thickness, of the implantable devices. In some examples, the implantable devices may be configured to deliver suitable ocular medicaments including but not limited to therapeutic agents, such as prostaglandin analogs (PGAs) (e.g., latanoprost), or therapeutic agents from other medicament classes, including beta-blockers such as timolol, alpha-2-agonists such as brimonidine tartrate, or carbonic anhydrase inhibitors such as dorzolamide, compounds of carbonic anhydrase inhibitors and beta-blockers, and compounds of alpha-agonists and beta-blockers which may be administered in combination with PGAs, for example.

In some embodiments, such implantable devices are configured to be implanted and have an internal volume that is adjustable one or more times, for example minimally invasively in situ without requiring removal of the device from an implantation site. Given the size and the subconjunctival and suprachoroidal target implantation locations, implantation procedures can be performed outside of the operating room, where needle puncture and small incisions are commonly performed. Such adjustment to the internal volume (e.g., increase or decrease in the volume) may help release the pressure within the eye as suitable if the initial volume of the implantable device applies too much pressure to the surrounding tissue, for example.

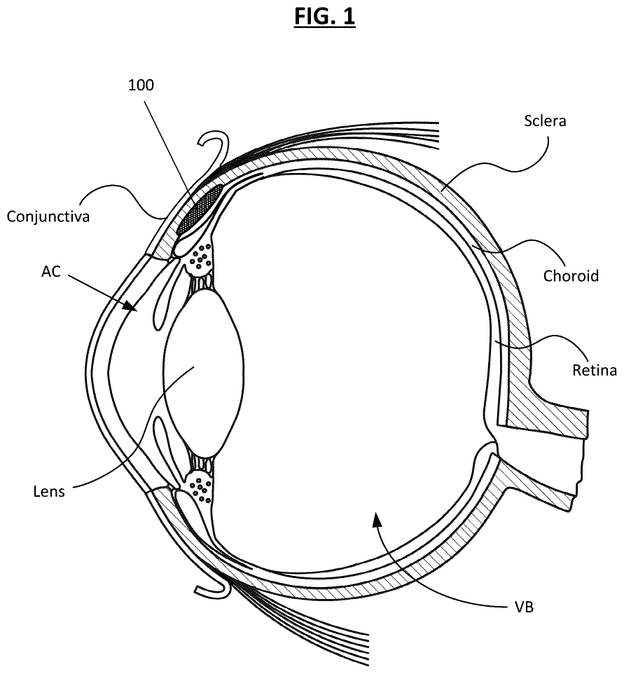

FIG. 1 illustrates an example of how a suprachoroidal implantable device 100 as disclosed herein may be implanted subconjunctivally and suprachoroidally within the eye, such as between the sclera and the choroid of the eye. Also shown are an anterior chamber (AC), a posterior chamber (PC), a choroid, a retina, a lens, and a vitreous body (VB) as relevant to the implant.

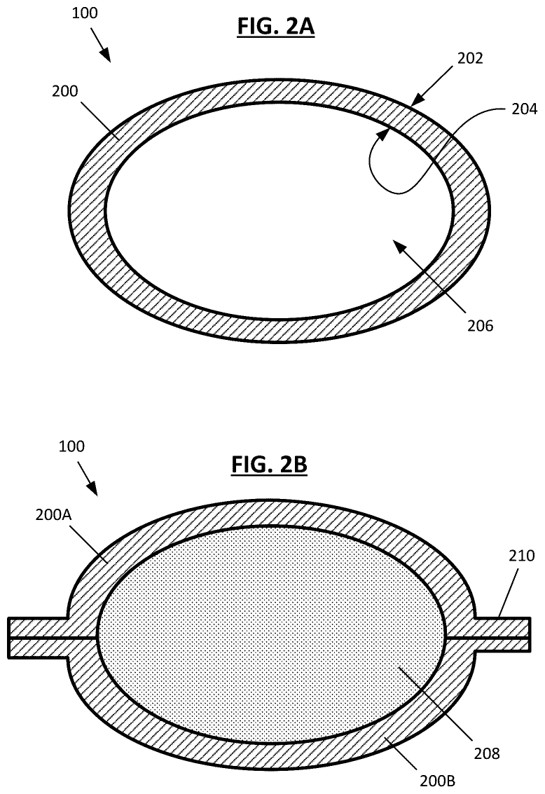

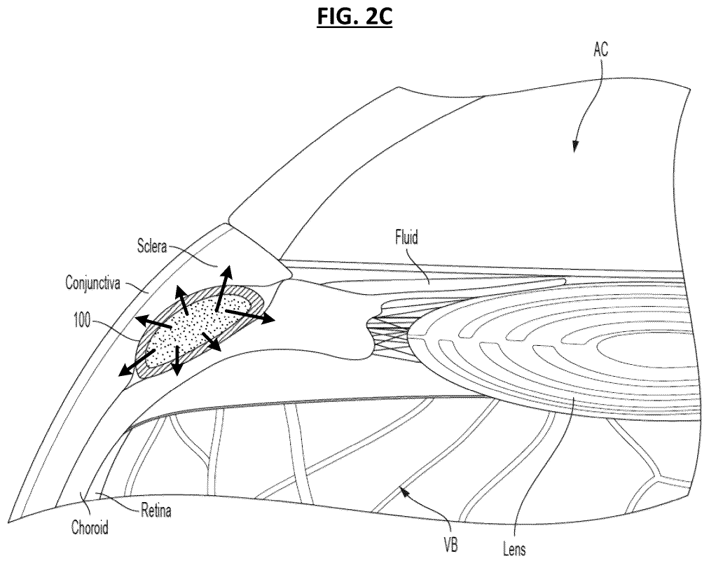

FIGS. 2A and 2B show examples of a suprachoroidal implantable device 100 according to embodiments disclosed herein. The device 100 includes a body or body portion 200 formed of a compliant material. The body portion 200 has an external surface 202, also described as a tissue ingrowth portion of the body thickness, and an opposing internal surface 204, also described as a boundary portion of the body thickness, which defines an internal reservoir 206 of the body portion 200. The internal reservoir 206 may have a fixed volume before and after implant, or an adjustable internal volume such that the volume can be adjusted by a surgeon or practitioner after the device 100 is implanted, for example by inserting a needle of a syringe through the body portion 200 to remove some of the fluid inside or to fill the reservoir 206 with additional fluid. In some examples, the body portion 200 is self-sealing such that the puncture hole is sealed shortly after the needle is removed. In some examples, the external surface 202 is visually observed to be less uniform than the internal surface 204 at a certain magnification, such as 100×, 200×, 500×, 1000×, or any other suitable value or range of magnification therebetween, as suitable. This difference in visual appearance may be a result of differences or variations in microstructure, including porosity. For example, the body portion 200 may have a variable porosity that transitions from a first porosity to a second porosity that is less than the first porosity. The first porosity is located proximate the external surface 202, and the second porosity is located proximate the internal surface 204. Thus, in some examples, the external surface 202 has a first porosity, and the internal surface 204 has a second porosity, where the second porosity may be less than the first porosity. The first porosity may facilitate tissue ingrowth at the external surface 202, and the second porosity may prevent or inhibit tissue ingrowth through the internal surface 204. The body portion 200 may be pre-sealed prior to the implant procedure to maintain a fixed volume of the reservoir 206.

In some examples, the ability for a surface to prevent or inhibit tissue ingrowth at a surface of the body portion as explained above may be observed via scanning electron microscopy (SEM). In some examples, a bubble point test may be performed to test the ability to prevent or inhibit tissue ingrowth. An example of such bubble point test method is disclosed in ASTM Testing Method F316-03 (“Standard Test Methods for Pore Size Characteristics of Membrane Filters by Bubble Point and Mean Flow Pore Test”). The bubble point test is based on the principle that a wetting liquid is held in these capillary pores by capillary attraction and surface tension, and the minimum pressure required to force liquid from these pores is a function of pore diameter. The pressure at which a steady stream of bubbles appears in this test is the bubble point pressure. For example, Table 1 of the aforementioned ASTM Testing Method F316-03 shows a general correlation between pore size and bubble point pressure with respect to the different fluids used. In some examples, a pore size of less than about 1 μm may be capable of preventing such tissue ingrowth.

In FIG. 2A, the body portion 200 is made of a single component, whereas in FIG. 2B, the body portion 200 is made of two components, 200A and 200B, that are adhered or attached together at a section such as a periphery 210 of the device 100. In some examples, the body portion 200 as shown in FIG. 2B may be implemented as a single component. The body portion 200 may be formed or manufactured via wrapping, rolling, or folding the single component into a suitable shape, and thereafter melted or sintered, for example to reduce or eliminate the seams in the body portion 200 from the wrapping, rolling, or folding of the single component. Also shown is a filler material 208 which may be contained or encapsulated within the reservoir 206 in order to maintain the internal volume of the device 100. In some examples, the body portion 200 is partially pre-filled with the filler material 208 to fill at least 10% of a maximum capacity of the internal volume of the internal reservoir 206. In some examples, the filler material 208 may pre-fill from about 10% to about 20%, from about 20% to about 30%, from about 30% to about 40%, from about 40% to about 50%, from about 50% to about 60%, from about 60% to about 70%, from about 70% to about 80%, or any other suitable value/range therebetween or combination of ranges thereof, with respect to the maximum capacity of the internal reservoir 206. For example, the filler material 208 may be any suitable fluid such as a hydrogel, a saline solution, or any suitable medicament as further explained herein, which may be selected to gradually pass through the compliant material from the internal surface 204 to the external surface 202 over a period of 3 days, 5 days, 7 days, 10 days, 15 days, 20 days, 25 days, 30 days, or any other suitable number or range therebetween, in order to leave the reservoir 206 to be dispersed in the surrounding environment within the eye, if so desired. In some examples, the filler material 208 may be a flexible polymeric material including but not limited to expanded polytetrafluoroethylene (ePTFE), for example. In some examples, the filler material may include but are not limited to: acrylamide, N-isopropylacrylamide, poly(methyacrylic-graft-ethylene glycol), cellulose, poly(ethylene oxide)-b-poly(propylene oxide)-b-poly(ethylene oxide) triblock copolymers (PEO-PPO-PEO), also known as poloxamers or Pluronics®, or polyacrylates. In some examples, the filler material may be biodegradable tissue-bulking agents including but not limited to collagen. In some examples, the filler material may include biocompatible tissue-bulking agents including but not limited to biocompatible gel such as silicone gel and/or calcium hydroxyapatite gel.

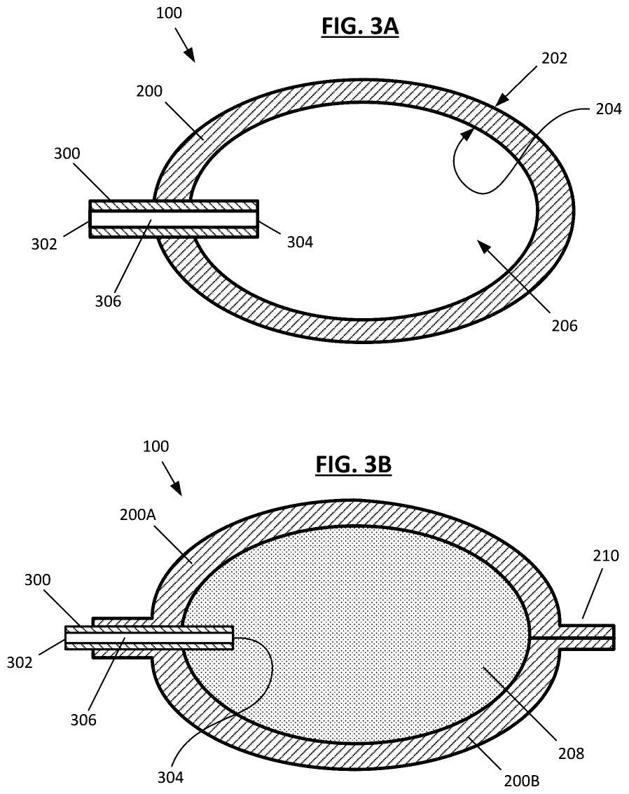

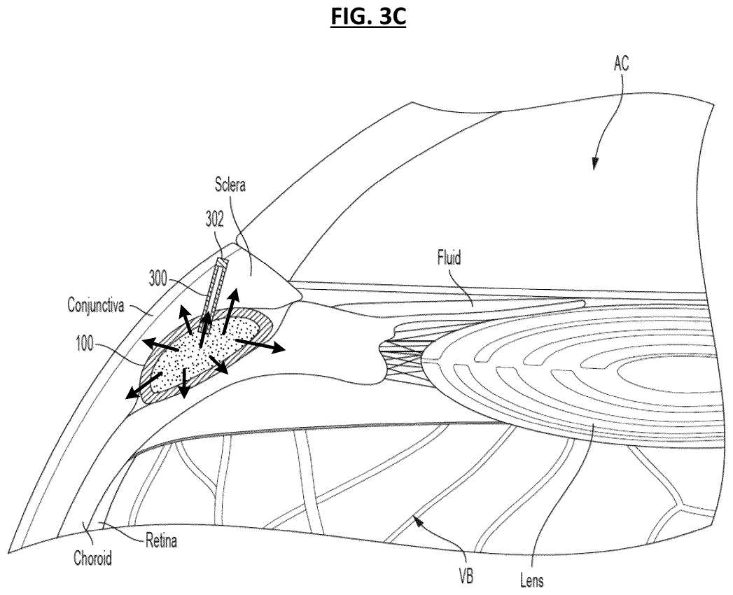

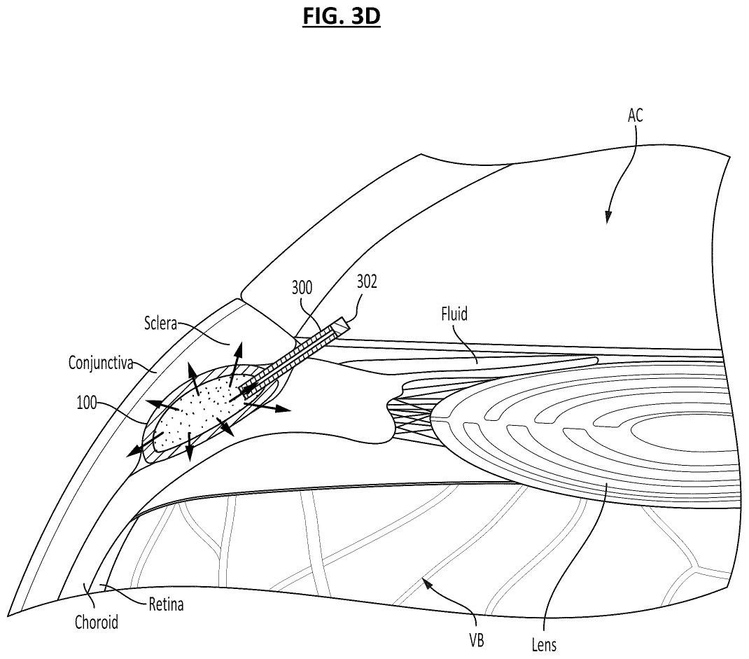

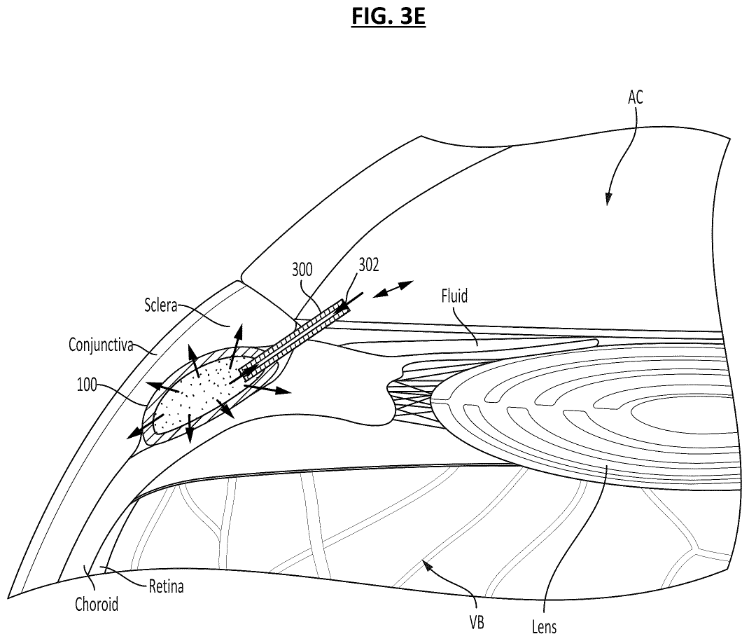

FIG. 2C shows how the device 100 of FIG. 2A or 2B may be implanted according to embodiments disclosed. The entirety of the device 100 may be implanted subconjunctivally in a suprachoroidal space in the eye, such as a space between the sclera and the choroid of the eye, or more specifically, the space underneath the sclera but above the choroid. In some examples, the device 100 as implanted maintains its fixed volume so as to keep applying a predetermined amount of pressure outwardly onto the surrounding tissue, such as the tissue of the sclera and/or of the choroid, thereby maintaining the space in an open configuration to allow fluid to flow into such space as formed. The fluid may be aqueous humor which, under normal conditions, is allowed to flow freely through the AC and exit through a drainage system within the eye, such as the trabecular meshwork. However, if the patient is suffering a condition which causes the drainage system of the eye to be blocked, the fluid can no longer flow freely, thus causing the IOP increases. As such, the space formed by the device 100 as shown is instead used as a secondary drainage pathway for the fluid (aqueous humor) to flow from the AC, in order to reduce the IOP within the eye. FIGS. 3C, 3D, 3E, 4D, 5D, and 17 similarly show the device 100, in different configurations as disclosed further herein, that facilitate the formation of such secondary drainage pathway for the fluid so as to reduce the IOP by providing the spacing as explained above. The possible directions of such fluid flow from inside the device 100 to an external surrounding are shown using arrows.

FIGS. 3A and 3B show examples of a suprachoroidal implantable device 100 according to various embodiments. The device 100, in addition to the body portion 200, surfaces 202 and 204, and reservoir 206 as mentioned above, further includes a sealable conduit 300 which has a first end 302 and a second end 304 opposing the first end 302, and the second end 304 is fluidly coupled with the internal reservoir 206 of the device 100 in order to facilitate in situ adjustment to the internal volume of the internal reservoir 206 by facilitating a fluid inflow into and a fluid outflow from the internal reservoir 206. The conduit 300 has a channel 306 extending therethrough from the first end 302 to the second end 304 to facilitate such adjustment.

In FIG. 3A, the conduit 300 is received (e.g., inserted) through the body portion 200. In FIG. 3B, the conduit 300 is sandwiched between the two components 200A and 200B of the body portion 200. In both cases, there may additionally be an adhesive component or other material disposed between an outer surface of the conduit 300 and the body portion 200 such that the conduit 300 remains in its location and does not move from its location.

FIG. 3C shows how the device 100 of FIG. 3A or 3B may be implanted according to embodiments disclosed. The body portion 200 of the device 100 may be implanted subconjunctivally in a suprachoroidal space in the eye as shown, and the conduit 300 may be disposed to extend through the scleral tissue. The first end 302 of the conduit 300 may be left open to fluidly couple the AC and the first end of the conduit 300 when the device 100 is being implanted to facilitate fluid communication therethrough from the internal surface 204 to the external surface 202 in response to a fluid pressure applied from the AC, and subsequently sealed as shown in FIG. 3C after the device 100 is implanted and the operator finishes adjusting in situ the internal volume of the device 100 to inhibit fluid communication therethrough, thereby maintaining the internal volume after the adjustment. The sealing may be performed by any suitable means including but not limited to blocking at least the first end 302 of the conduit 300 via melting or sintering. As disclosed herein, the conduit 300 may be formed of any suitable material such as polymer with a predetermined melting point to facilitate such means of sealing the first end 302. The possible directions of such fluid flow from inside the device 100 to an external surrounding as well as into the conduit 300 are shown using arrows.

FIGS. 3D and 3E show configurations in which the device 100 may be implanted such that the conduit 300 extends toward the AC, instead of being disposed through the scleral tissue. In FIG. 3D, the first end 302 of the conduit 300 is sealed as explained above, and the possible directions of such fluid flow from inside the device 100 to an external surrounding as well as into the conduit 300 are shown using arrows, whereas in FIG. 3E, the first end 302 remains open to allow the fluid from the AC (such as the aqueous humor) to flow through the conduit 300 (or more specifically, through the channel 306 of the conduit 300) from the first end 302. The flow of fluid may be bidirectional as shown by the double-headed arrow in FIG. 3E. That is, at any time, fluid may either flow from the outside of the device 100 entering the conduit 300, or exit the device 100 from the reservoir 206 to the surrounding tissue within the eye. Therefore, the device 100 is capable of self-adjusting its own internal volume based at least partially upon a change in the surrounding environment, or more specifically a change in the fluid pressure surrounding the device 100.

In some embodiments, one or more of the surfaces (e.g., the first or external surface 202 and/or the second or internal surface 204) may include a microporous microstructure. For example, one or more of the surfaces may include biocompatible materials such as ePTFE. Additionally, one or more of the surfaces may be formed of other biocompatible materials including biocompatible polymers, which may or may not be microporous, including, but not limited to, polyurethane, silicone, polysulfone, polyvinylidene fluorine (PVDF), polyhexafluoropropylene (PHFP), perfluoroalkoxy polymer (PFA), polyolefin, fluorinated ethylene propylene (FEP), acrylic copolymers, and polytetrafluoroethylene (PTFE).

One or more of the aforementioned surfaces may be in the form of one or more sheets or films, and they may include knitted, woven, and/or non-woven forms including individual or multi-fiber strands. In some embodiments, the surface(s) may be formed from a plurality of sheets or films of polymer material. In some embodiments, the sheets or films may be laminated or otherwise mechanically coupled together to form the surface(s) as well as to form the body portion of the implantable device, such as the body portion 200. Coupling of the sheets or films may be accomplished by a variety of mechanisms, including heat treatment, high pressure compression, bonding agents such as one or more adhesives, lamination, or other suitable methods known to one of skill in the art.

In some embodiments, adjacently-situated surfaces (e.g., the surfaces 202 and 204) and/or the layers of material forming such surfaces, may be partially or completely bonded or adhered in any of a variety of manners. For example, in some examples the layers are bonded via thermal methods where each of the polymers forming the materials are brought to or above their melting temperatures. In some embodiments, such thermal processes facilitate adhesive or cohesive bond formation between the materials or layers of material. In some embodiments, adjacently situated surfaces and/or the layers of material forming such surfaces, may be partially bonded via thermal methods where at least one of the materials is brought to or above its melting temperature. Such thermal processes may facilitate adhesive or cohesive bond formation between the materials or layers of material. In some embodiments, one or more suitable adhesives are utilized and provide a sufficiently bonded interface. Adjacently situated surfaces and/or the layers of material forming such surfaces, may be coupled together at one or more discrete locations such as the periphery (e.g., periphery 210) to form stabilizing structures that extend through the resulting structure.

In some examples, the tube or conduit (e.g., the sealable conduit 300) and/or the filler material 208 may comprise material(s) including but not limited to: PTFE, ePTFE, urethanes, polyurethane, silicones (organopolysiloxanes), polysulfone, PVDF, PHFP, PFA, polyolefin, FEP, ethylene fluorinated ethylene-propylene (EFEP), ethylene-tetrafluoroethylene (ETFE), 3′-(2-aminopyrimidyl)-2,2′:5′,2″-terthiophene (PATT), and acrylic copolymers, among others. In some embodiments, the materials may include other biocompatible polymers suitable for use in forming any one or more of the tube or conduit including, but not limited to, copolymers of silicon-urethane, styrene/isobutylene copolymers, polyisobutylene, polyethylene-co-poly(vinyl acetate), polyester copolymers, nylon copolymers, fluorinated hydrocarbon polymers and copolymers or mixtures of any of the foregoing may be used. In various embodiments, the elastomer or elastomeric material may include perfluoromethyl vinyl ether and tetrafluoroethylene, (per) fluoroalkylvinylethers (PAVE), a copolymer of tetrafluoroethylene and perfluoromethyl vinyl ether, silicone, a fluoroelastomer, a urethane, butyl rubber, styrene-butadiene, isobutylene-isoprene, or a TFE/PMVE copolymer.

In some examples, the device 100 may include one or more portions that are configured to promote or permit cellular infiltration and/or tissue attachment. The device may also deliver medicament which may include a single therapeutic agent (e.g., a medication), or may include multiple therapeutic agents. The medicament may include additional materials (e.g., bioabsorbable polymers, pharmaceutically acceptable carrier) to affect the elution of the therapeutic agents (e.g., bioabsorbable polymers) from the delivery device. Throughout the description herein, the medicament may also be referred to as a drug or a pharmaceutical composition or combination as is may be composed of both a therapeutic agent and/or additional materials for effective elution of the therapeutic agent. For example, the medicament may include bioabsorbable microparticles having a size ranging between approximately 0.1 microns to 50 microns, or from approximately 1 micron to 50 microns, or from approximately 5 microns to 50 microns, or from approximately 15 microns to 50 microns, or from approximately 10 microns to 40 microns, or approximately from 15 microns to 25 microns, or approximately 18 microns to 23 microns. In some embodiments, the bioabsorbable microparticles have an average size of approximately 20 microns. In further embodiments, the therapeutic agent retained in the bioabsorbable microparticles may be latanoprost. The bioabsorbable microparticles may be retained within the device 100 during use, while the medicament may be released from the bioabsorbable microparticles, and as such, the device 100.

In some examples, the medicament may include anti-inflammatory medicaments which are typically used in post-operation, including but not limited to: dexamethasone, prednisolone, ketorolac, nepafenac, bromofenac, diclofenac, cyclosporine, or lifitegrast. In some examples, the medicament may include antibiotics typically used in post-operation, including but not limited to moxifloxacin or gatifloxacin. In some examples, when the medicament delivery system is so arranged to treat glaucoma, API classes can include prostaglandins such as latanoprost, bimatoprost, and latanoprost (e.g., XALATAN®). In certain instances, active pharmaceutical ingredient (API) classes can include beta blockers such as timolol (e.g., Betimol®); alpha agonist such as brimonidine or Alphagan®; or carbonic anhydrase inhibitors such as dorzolamide, brinzolamide (e.g., Azopt®); or miotics such as pilocarpine. API classes can include monoclonal antibodies including but not limited to: bevacizumab (e.g., Avastin®), ranibizumab (e.g., Lucentis®), or aflibercept (e.g., Eylea®).

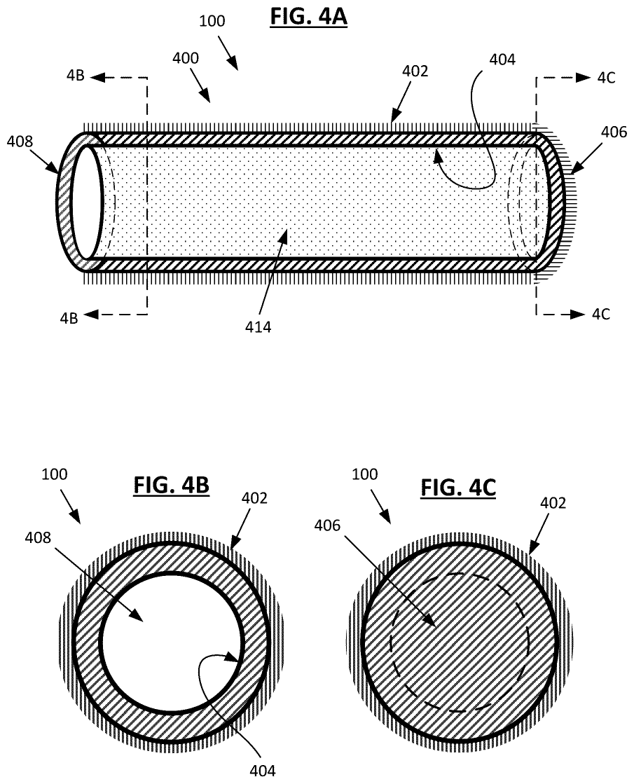

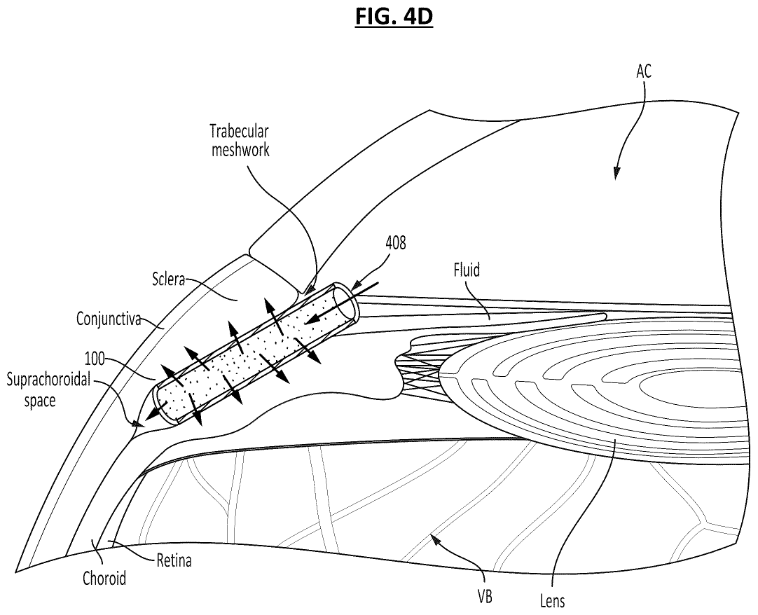

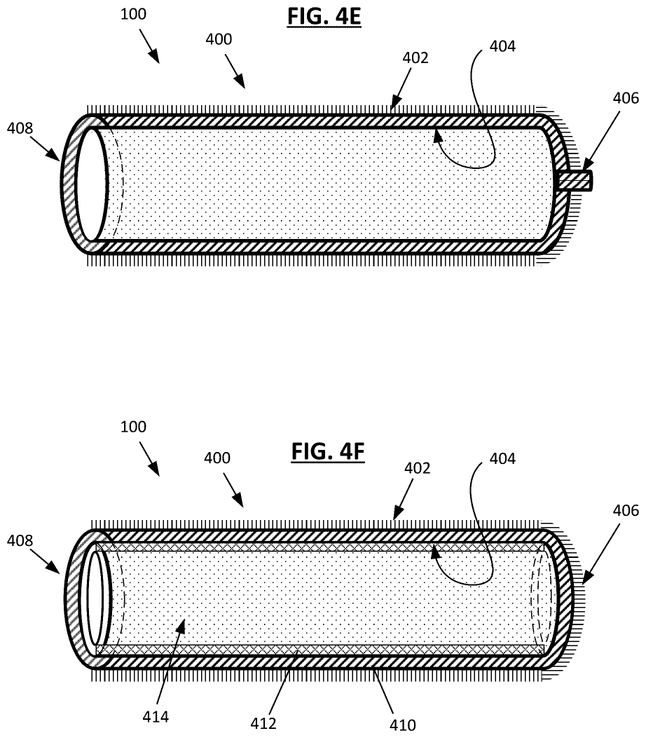

FIGS. 4A through 4C show an example of a suprachoroidal implantable device 100 according to embodiments disclosed herein. The device 100 includes a body or body portion 400 with a closed end 406 and an open end 408 that is fluidly coupled with the AC of the eye when implanted. According to examples, the device 100, or the body 400 thereof, may have a substantially circular or round cross-section. The body portion 400 includes a first surface 402, also described as a tissue ingrowth portion of the body thickness, and a second surface 404, also described as a boundary portion of the body thickness, which may be similar in material, physical structure, or properties with respect to the external surface 202 and the internal surface 204, respectively, previously described. That is, in some examples, the second surface 404 may be less uniform than the first surface 402 when observed at a certain magnification, such as 50×, 100×, 200×, 500×, 1000×, or any other suitable value or range of magnification therebetween, as suitable. The body portion 400 may be pre-formed as a cup-like construct, that is, a substantially cylindrical construct with a base that is closed to form the closed end 406 while the other end is open to form the open end 408. Also, in some examples, the body portion 400 may have a variable porosity that transitions from a first porosity to a second porosity that is less than the first porosity. The first porosity is located proximate the first surface 402, and the second porosity is located proximate the second surface 404. In some examples, the first surface 402 has a first porosity, and the second surface 404 has a second porosity less than the first porosity. The first porosity may facilitate tissue ingrowth at the first surface 402, and the second porosity may prevent or inhibit tissue ingrowth through the second surface 404.

FIG. 4D shows an example in which the device 100 of FIGS. 4A through 4C may be implanted such that the open end 408 of the body portion 400 is in fluid communication with the AC, for example, to facilitate unidirectional fluid flow from the AC to an internal volume of the device 100, and from the internal volume of the device 100 to the external surrounding such as within the suprachoroidal space. The body portion 400 may be substantially cylindrical or tubular in shape or construct, the substantially cylindrical or tubular construct may define an internal volume or internal space 414 of the body portion 400. The internal volume or space 414 may be an open volume, that is, a volume that is not sealed from the surrounding environment. The construct may be self-supporting to maintain the open volume regardless of whether the volume is empty or filled (partially or entirely) with fluid such as aqueous humor. The possible directions of such fluid flow from inside the device 100 to an external surrounding are shown using arrows, with little to no outflow through the open end 408 due to the difference in pressure within the AC of the eye. The unidirectional fluid flow may be facilitated by the intraocular pressure within the AC of the eye being at or above a threshold pressure level, for example 20 mm Hg, 30 mm Hg, 40 mm Hg, 50 mm Hg, or any other suitable value or range therebetween.

FIG. 4E shows a generalized view of an example of the device 100 in which the closed end 406 of the body portion 400 is formed by pinching and closing one end of a material that is originally formed as a tubular construct. After pinching an end of the tubular construct, the end may be melted or sintered such that the pinched end remains permanently closed to form the closed end 406.

FIG. 4F shows an example of the device 100 in which the body portion 400 includes an external microporous layer 410 and an internal elastic support structure 412. The microporous layer 410 defines the first surface 402 and the second surface 404 as described above, and the support structure 412 is disposed within the microporous layer 410 and defines the internal volume or space 414. The support structure 412 may have a third porosity that is different from the first and second porosities as explained above. For example, the third porosity may be greater than the first porosity and the second porosity to facilitate fluid communication between the internal volume or space 414 and the second surface 404. This may allow the fluid which enters the internal volume or space 414 of the body portion 400 to flow outwardly through the first surface 402 and into the surrounding environment. In some examples, the open end 408 may receive fluid into the internal volume or space 414, and the closed end 406 may allow the received fluid to flow outwardly and leave the internal volume or space 414. In some examples, an external surface of the body portion 400 may be defined by the first surface 402 while an internal surface of the body portion 400 may be defined by the second surface 404.

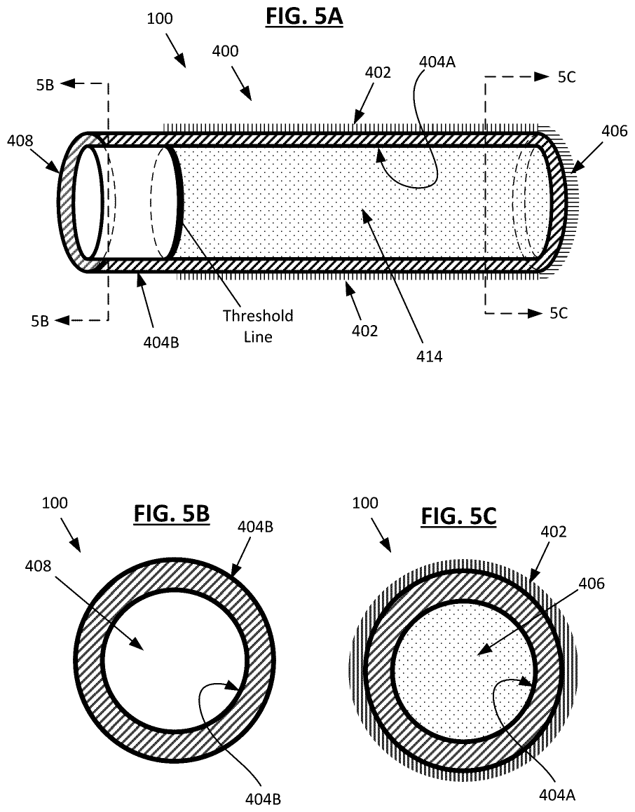

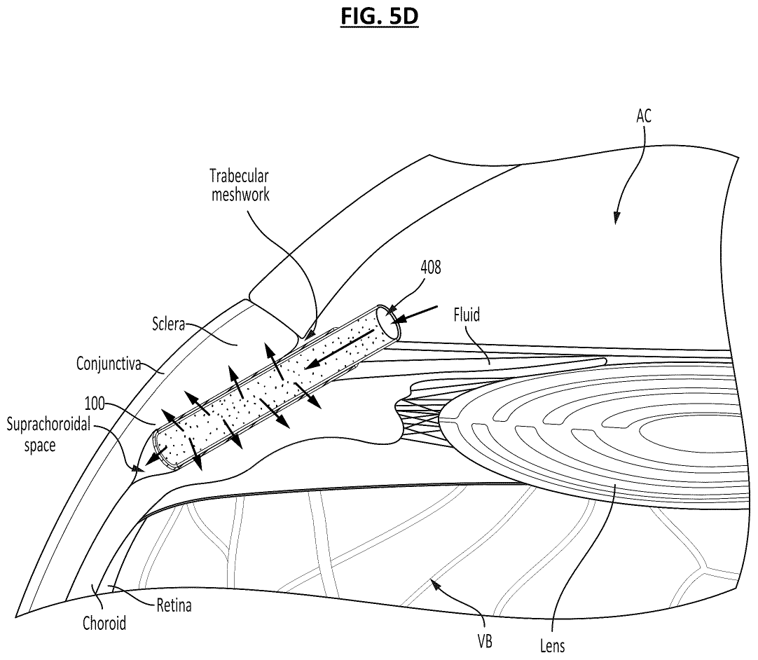

FIGS. 5A through 5C show an example of a suprachoroidal implantable device 100 according to embodiments disclosed herein. The device 100 includes a body or body portion 400 being formed of a material having a thickness defining an ingrowth portion that facilitates tissue ingrowth and a boundary portion that prevents or inhibits tissue ingrowth. The ingrowth portion defines at least a portion of the external surface or external region of the body 400 that directly contacts an external tissue within the eye, and the boundary portion defines at least a portion of the internal surface or internal region of the body 400. The boundary portion (or the second surface 404) has two sections: a first section 404A which covers an entirety of the internal surface of the body portion 400 and a second section 404B which covers a portion of the external surface of the body portion 400, for example until the “threshold line” as labeled on FIG. 5A. As such, a portion of the external surface of the body portion 400 may be covered or defined by the ingrowth portion (or the first surface 402) while the remaining portion of the external surface of the body portion 400 may be covered or defined by the boundary portion (or the second section 404B of the second surface 404). The internal surface of the body portion 400 is defined solely by the boundary portion (or the first section 404A of the second surface 404). The second section 404B of the second surface 404 may define about 10%, 20%, 30%, 40%, 50%, 60%, 70%, 80%, 90%, or any other suitable range or number therebetween, of the external surface of the body portion 400, while the remaining percentage of the external surface is defined by the first surface 402. The portion of the external surface that is defined by the ingrowth portion (or the first surface 402) may appear less uniform when inspected at a first magnification than the portion of the internal surface that is defined by the boundary portion (or the second surface 404). The first magnification may be 50×, and optionally 100×, and optionally 500×, and optionally 1000×.

FIG. 5D shows an example in which the device 100 of FIGS. 5A through 5C may be implanted such that the second section 404B of the second surface 404 extends into the AC such that only the portion of the external surface of the device 100 that is defined by the first surface 402 is disposed in the suprachoroidal space, or between the choroid and the sclera, such that the first surface 402 facilitates tissue ingrowth from the scleral tissue and the choroidal tissue, while the second section 404B of the second surface 404 prevents or inhibits such tissue ingrowth, such that the device 100 may facilitate unidirectional fluid flow from the AC to an internal volume of the device 100, and from the internal volume of the device 100 to the external surrounding such as within the suprachoroidal space. The possible directions of such fluid flow from inside the device 100 to an external surrounding are shown using arrows, with little to no outflow through the open end 408 due to the difference in pressure within the AC of the eye. The unidirectional fluid flow may be facilitated by the intraocular pressure within the AC of the eye being at or above a threshold pressure level, for example 20 mm Hg, 30 mm Hg, 40 mm Hg, 50 mm Hg, or any other suitable value or range therebetween.

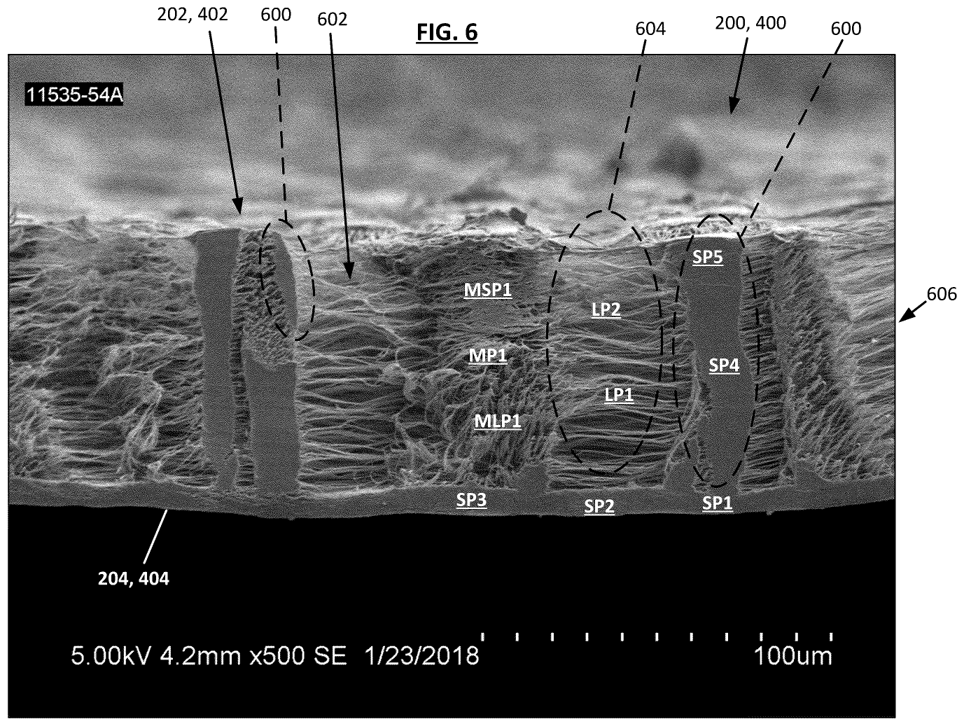

FIG. 6 shows a microscopic view of a microporous material of the body portion 200 or 400 of the suprachoroidal implantable device 100 according to some embodiments. Displayed at the bottom of FIG. 6 is: “5.00 kV 4.2 mm×500 SE Jan. 23, 2018,” and the distance between two consecutive lines as shown at the bottom right hand corner represents 10 μm. For example, the microporous material of FIG. 6 may be referred to throughout with reference to an implantable device or system. As can be appreciated by a person of skill in the art and with reference to FIG. 6, the microporous aspects and parameters of the microporous material can be defined in a variety of ways. In an application of a microporous material in an ocular device, such as the implantable device 100 described herein, configured for in situ placement in the tissue of the eye to control an internal fluid pressure of the eye, the microporous properties of such a microporous material can be generally characterized by a volumetric porosity value that can be defined as a ratio of a volume of the air or fluid defined by and contained within the microporous material as compared to an overall volume (or total volume) of the microporous material.

In another definition, a volumetric porosity can be defined as a percentage of the microporous material volume that is occupied by non-structural or transient elements such as air or other fluids. For example, a microporous material with an overall volume of 100 mm³ and with 30 mm³ of that volume comprising chambers holding air or a fluid would have a volumetric porosity value of 0.3 because 30% of the volume of the microporous material is empty or transient space that is filled with air or other fluids.

As can be appreciated, two microporous materials can have the same volumetric porosity but differ in the pore sizes presented to the incoming or exiting air or fluid. For example, a first material can a have a small number of large pores distributed over a fixed overall volume and a second material can have a relatively large number of relatively smaller pores distributed over the same fixed volume, and both microporous materials could have the same volumetric porosity if the air/fluid volume of the two materials are the same.

As can be further appreciated, the properties of the microporous materials used in the device can also be defined by the size of the passages passing through the microporous material or similarly defined as a pore size measured where a passage terminates at a surface of the microporous material or measured along a length of a passage within the material. Microporous materials with small pores or passages can impede flow through the material and comparatively large pores or passages can provide an increased pass through of the air or fluid into, out of, or within the microporous material.

As can be still further appreciated, the properties of the microporous material can also be defined by a tortuosity of the passages entering into and passing through the material, with relatively small or large passages presenting impeded fluid pathways due the frequency of turns in the passages or by the placement of obstructions in the fluid pathways. The air/fluid passthrough rates of a microporous material can be managed by controlling or defining any of the above-described characteristics of the material to provide a suitable material for use to facilitate pressure control in the eye for the treatment of a disease.

For simplicity, the aforementioned characteristics and variables of the microporous material used in the various embodiments and examples described herein can be presented simply as a porosity which can be based on a volumetric porosity, a pore or passage size, and/or a tortuosity metric. Again, with reference to FIG. 6, internal portions of the microporous material can have varying porosity or porosities (or volumetric porosities, or pore sizes, and/or tortuosities). The internal portions can extend between an internal surface 204 (or second surface 404) and the external surface 202 (or first surface 402).

At any of these portions of a body portion 200 or 400, the porosity can comparatively range in degree from small pore size (SP), medium-small pore size (MSP), medium pore size (MP), medium-large pore size (MLP), and large pore size (LP), where LP is larger than MLP, MLP is larger than MP, MP is larger than MSP, and MSP is larger than SP. In some examples, the size of SP may range from about 0.01% to 2%, MP may range from about 2% to 20%, and MLP may range from about 20% to 80% that of the size of LP. The size of SP may range from about 0.01 μm to about 1 μm in pore size (as measured as pore diameter or pore average dimension). In some examples, the pore diameter may be a maximum diameter measured across a pore or a maximum cross-sectional length of a pore, and the pore average dimension may be the calculated average of different dimensional lengths as measured across the pore. In some examples, the porosity may increase by about 5 to 10 times as the pore size increases from one category to the next category (for example, from SP to MSP or from MSP to MP, etc.). Assuming, for discussion purposes here, that delivery travels along a relatively straight path through a microporous material so as to sequentially engage porosities of the internal surface 204 or second surface 404, a uniform internal portion, and the external surface 202 or first surface 402, the combined flow resistance can be represented by likewise concatenating their respective porosities. For instance, the internal surface 204 or second surface 404 typically has a low porosity throughout (e.g., to resist tissue ingrowth into the reservoir 206), and portions of the interior portions and the external surface 202 or first surface 402 can have any of the aforementioned degrees of porosity. Under these circumstances when the internal portion has a medium porosity and, for example, the internal portions have a medium porosity and the external surface 202 or first surface 402 has a high porosity, fluid may be delivered through the microporous material from the reservoir 206, for example, to tissue surrounding the device can be represented as SP-MP-LP. More examples are discussed here below.

Various delivery paths can be present within the microporous material. Relatively linear flow paths may comprise regions SP1-SP4-SP5, for example or SP3-MLP1-MP1-MSP1. Although some flow paths may be relatively straight, there are also flow paths that are nonlinear. For instance, under certain conditions, at least some flow may proceed to flow through areas of increasingly less resistance such as SP1-LP1-LP2 or SP3-MLP1-LP1-LP2. As will be appreciated, the microstructure of the microporous materials may undergo modification processes to obtain certain types of flow through the microstructure. For instance, the microstructure may have relatively uniform layers across layered within the microstructure, or as shown here, have variable portions throughout the thickness of the microporous material.

In some examples, the body portion 200 or 400 defines a wall portion thickness extending between the internal surface 204 or second surface 404 (also referred to as a boundary portion) and the external surface 202 or first surface 402 (also referred to as an ingrowth portion). The wall portion thickness can define an intermediate or transition portion 606 of the body portion 200 or 400 located between the boundary portion and the ingrowth portion, the transition portion 606 having a transition porosity that is between a porosity of the low porosity surface (e.g., having smaller pore sizes) of the internal surface 204 or second surface 404 (boundary portion of the thickness) and a porosity of the high porosity surface (e.g., having larger pore sizes) of the external surface 202 or first surface 402 (tissue ingrowth portion of the thickness). In addition, or in alternative, the transition portion 606 can have a transition portion porosity that is equal to porosities of the low porosity surfaces of the internal surface 204 or second surface 404 and the external surface 202 or first surface 402. In addition, or in alternative, the transition portion 606 can have a transition portion porosity that is equal to a porosity of the low porosity surface of the internal surface 204 or second surface 404. In addition, or in alternative, the transition portion 606 can have a transition portion porosity that is equal to a porosity of the high porosity surface of the external surface 202 or first surface 402.

In some examples, the body portion 200, 400 includes a plurality of nodes 600 and fibrils 602. A node may be any section of the external surface 202 or first surface 402 which has a “clump” or larger volume of polymer than a fibril. The node, in some examples, includes sections of the body portion 200, 400 with the small pore size (SP) as explained above with regard to FIG. 6, for example SP4 and SP5 as circled. The body portion 200, 400 is thus formed of a plurality of such nodes and fibrils that are interconnected, interlocked, or interweaved with each other, as appropriate. In some examples, the nodes have different sizes and porosities, and the fibrils may define spaces or openings 604 having the larger pore sizes (LP), such that the pore sizes of the section defined by the fibrils are larger than the pore sizes of the nodes. In some examples, the external surface 202 or first surface 402 may have spaces or openings 604 (for example, circled around LP1 and LP2) formed between neighboring nodes 600 and defined by the plurality of fibrils 602. In some examples, a size of the openings or spaces 604 may be defined by a distance between neighboring nodes 600 (e.g., an internodal distance). In some examples, the surface feature of the surface 202, 402 includes solid portions (e.g., the nodes 600 and fibrils 602, as well as any other solid material or object defining the structure of the body portion 200, 400) and pore portions (e.g., the internodal areas defining the size of the openings or spaces 604 which may be defined by the solid material or object defining the structure of the body portion 200, 400). The pore portions may include pores of 5 μm to 100 μm in size which are evenly distributed between the solid portions. In some examples, the size of such pores may be from about 1 μm to about 5 μm, from about 5 μm to about 10 μm, from about 10 μm to about 15 μm, from about 15 μm to about 20 μm, from about 20 μm to about 30 μm, from about 30 μm to about 40 μm, from about 40 μm to about 50 μm, from about 50 μm to about 60 μm, from about 60 μm to about 70 μm, from about 70 μm to about 80 μm, from about 80 μm to about 90 μm, from about 90 μm to about 100 μm, or any other suitable value/range therebetween or combination of ranges thereof. In some examples, the pore portions are flexible (e.g., the solid portions are made of a flexible material) to allow expansion of the pores, such that the size of the pores may vary, for example under physiologic conditions.

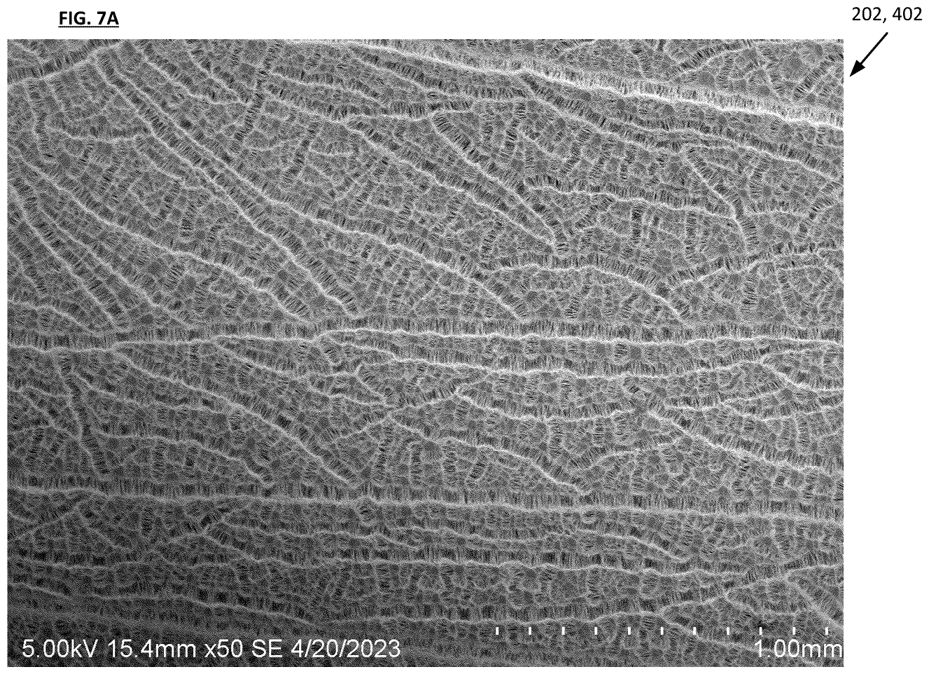

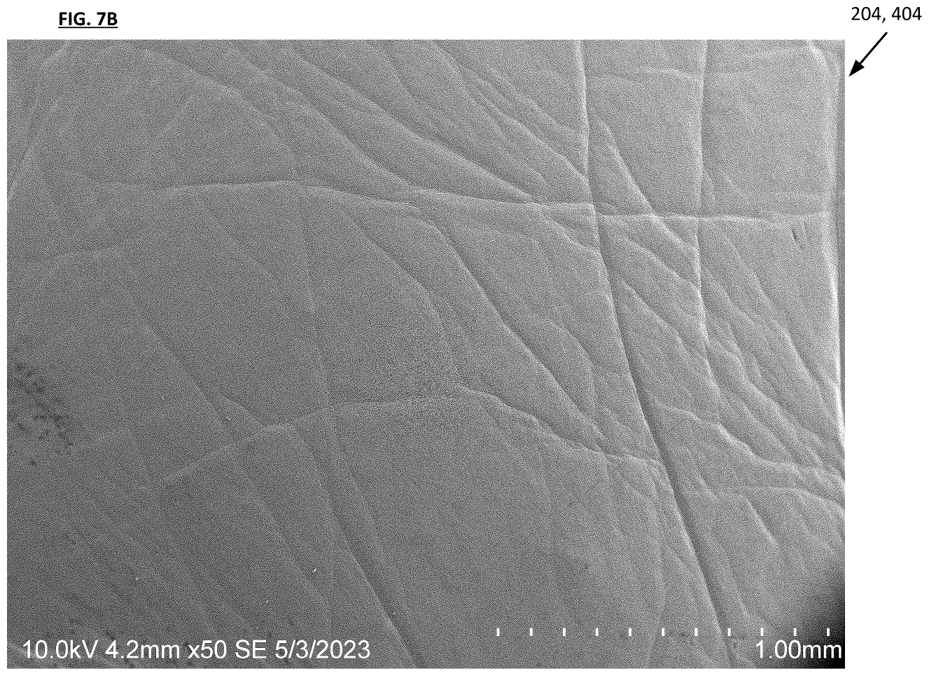

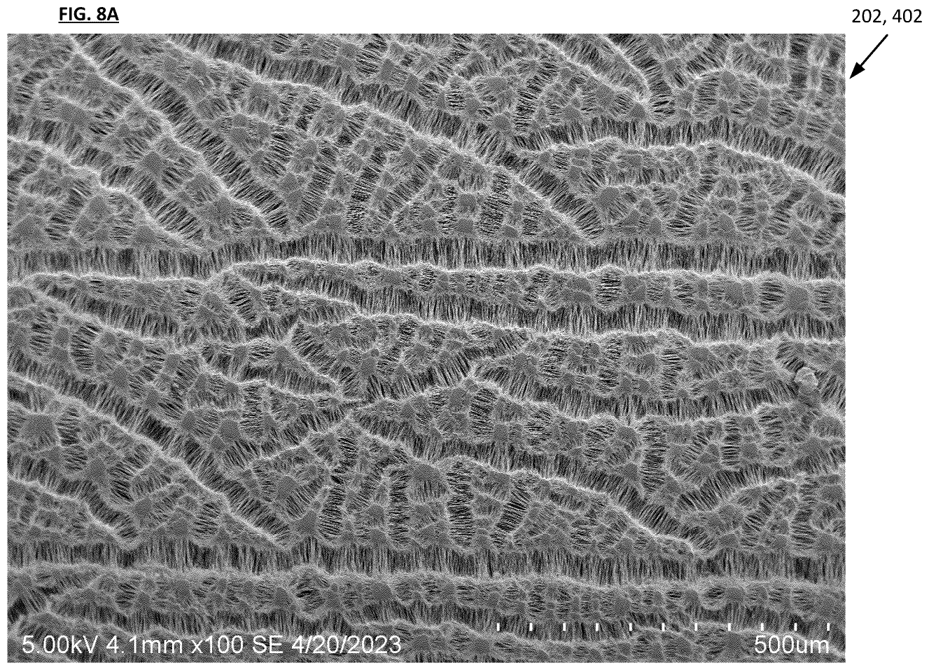

FIGS. 7A and 7B show a comparison of SEM images taken of the surfaces at the same 50× magnification. Displayed at the bottom of FIG. 7A is: “5.00 kV 15.4 mm×50 SE Apr. 20, 2023,” and the distance between two consecutive lines as shown at the bottom right hand corner represents 0.1 mm. Displayed at the bottom of FIG. 7B is: “10.0 KV 4.2 mm×50 SE May 3, 2023,” and the distance between two consecutive lines as shown at the bottom right hand corner represents 0.1 mm. FIG. 7A shows the surface 202, 402. FIG. 7B shows the surface 204, 404. Other possible magnifications may be 100×, 200×, 500×, 1000×, or any other suitable value or range of magnification therebetween, for example, as shown in additional figures as described herein.

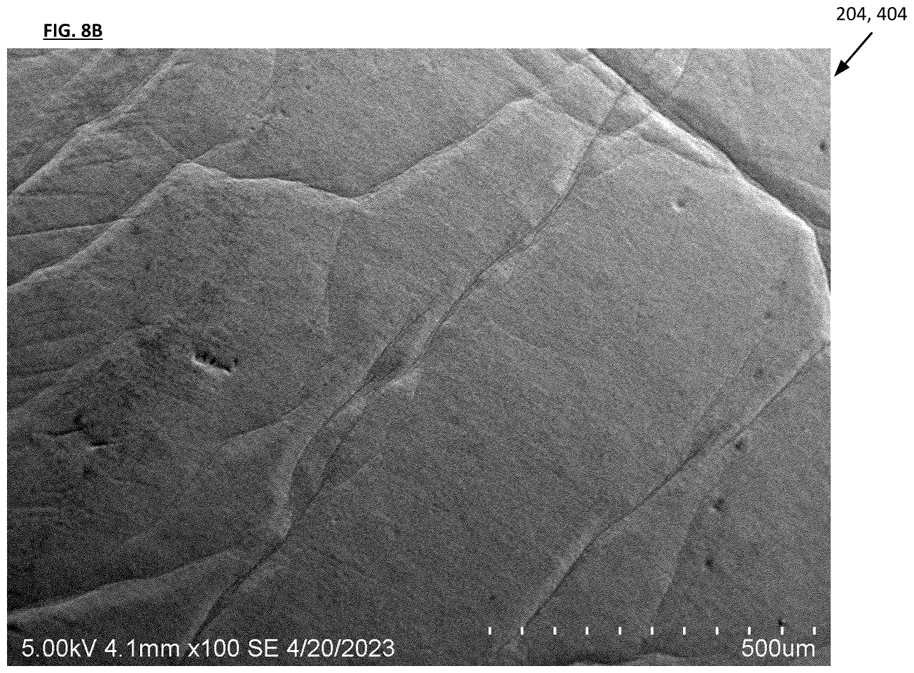

FIGS. 8A and 8B show a comparison of SEM images taken of the surfaces at the same 100× magnification. Displayed at the bottom of FIG. 8A is: “5.00 kV 4.1 mm×100 SE Apr. 20, 2023,” and the distance between two consecutive lines as shown at the bottom right hand corner represents 50 μm. Displayed at the bottom of FIG. 8B is: “5.00 kV 4.1 mm×100 SE Apr. 20, 2023,” and the distance between two consecutive lines as shown at the bottom right hand corner represents 50 μm. FIG. 8A shows the surface 202, 402. FIG. 8B shows the surface 204, 404.

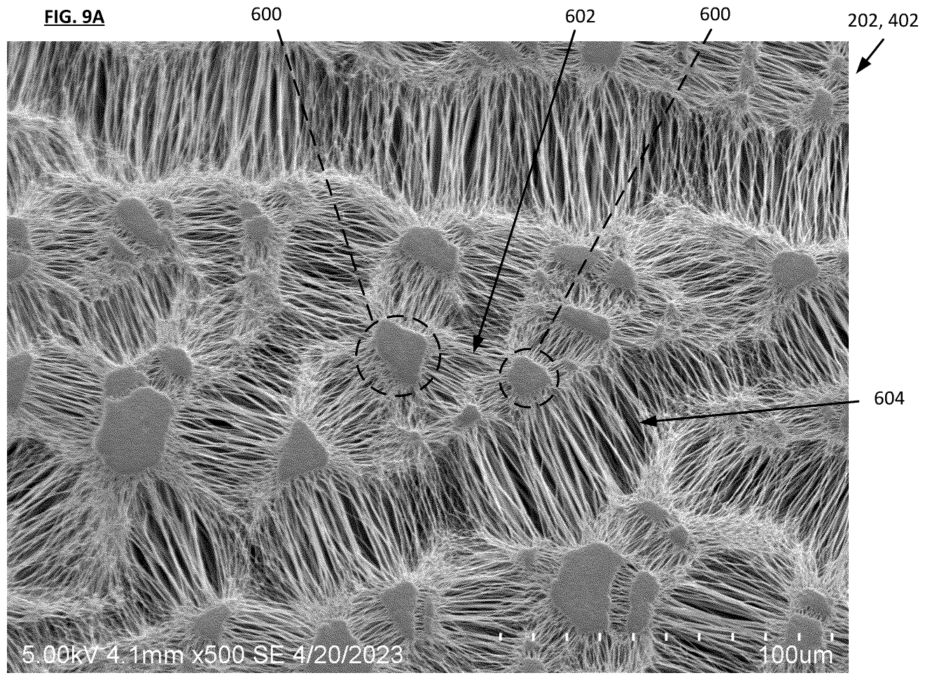

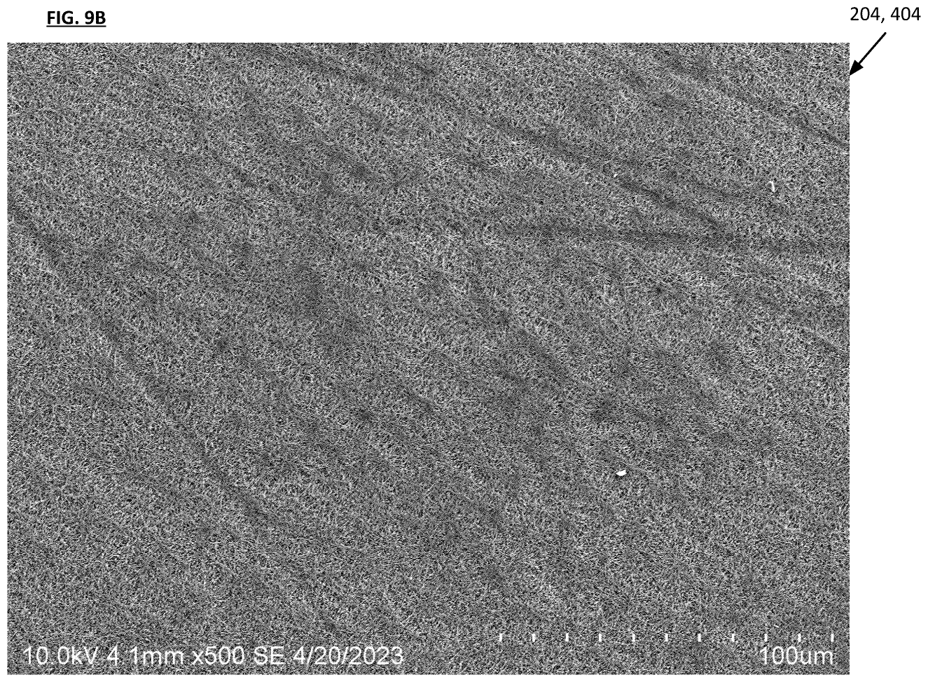

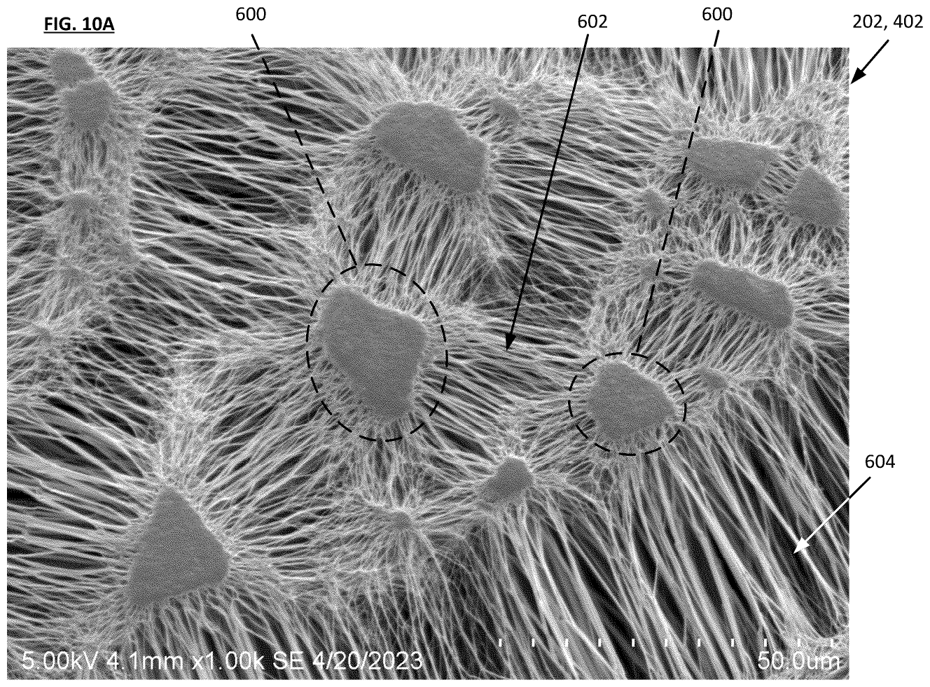

FIGS. 9A and 9B show a comparison of SEM images taken of the surfaces at the same 500× magnification. Displayed at the bottom of FIG. 9A is: “5.00 kV 4.1 mm×500 SE Apr. 20, 2023,” and the distance between two consecutive lines as shown at the bottom right hand corner represents 10 μm. Displayed at the bottom of FIG. 9B is: “10.0 kV 4.1 mm×500 SE Apr. 20, 2023,” and the distance between two consecutive lines as shown at the bottom right hand corner represents 10 μm. FIG. 9A shows the surface 202, 402. FIG. 9B shows the surface 204, 404.

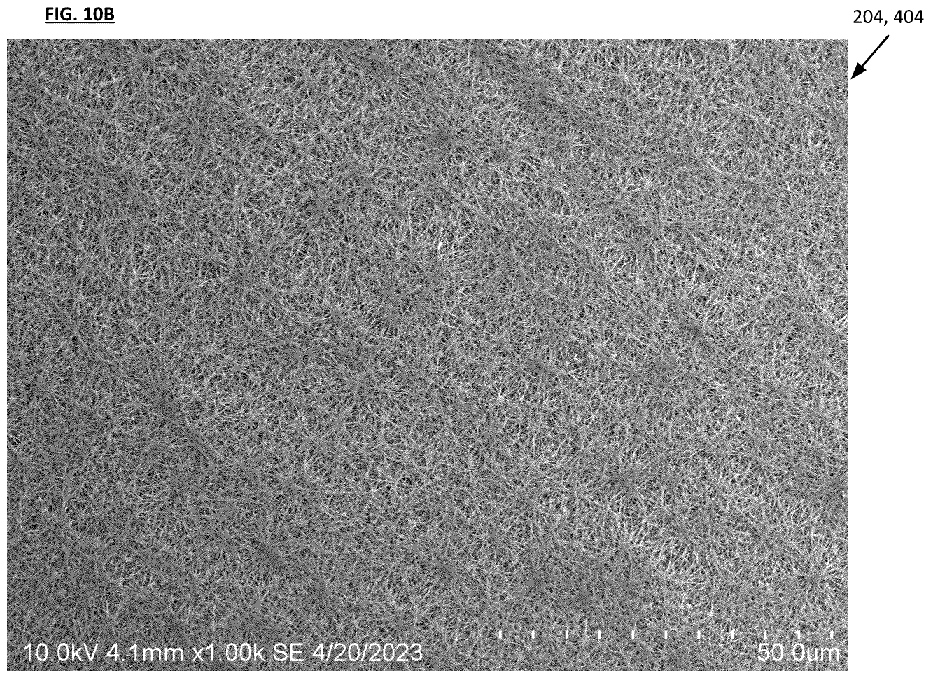

FIGS. 10A and 10B show a comparison of SEM images taken of the surfaces at the same 1000× magnification. Displayed at the bottom of FIG. 10A is: “5.00 kV 4.1 mm×1.00 k SE Apr. 20, 2023,” and the distance between two consecutive lines as shown at the bottom right hand corner represents 5 μm. Displayed at the bottom of FIG. 10B is: “10.0 kV 4.1 mm×1.00 k SE Apr. 20, 2023,” and the distance between two consecutive lines as shown at the bottom right hand corner represents 5 μm. FIG. 10A shows the surface 202, 402. FIG. 10B shows the surface 204, 404.

In at least one of the aforementioned magnifications, any one or more of the features mentioned below in the surfaces, hereinafter referred to as “surface features”, may be observed, when each of the surfaces that are being compared is viewed at the same magnification. In some examples, such features are observed more in the SEM images of the external surface 202 or first surface 402 (FIGS. 7A, 8A, 9A, and/or 10A) than in the internal surface 204 or second surface 404 (FIGS. 7B, 8B, 9B, and/or 10B). In some examples, such features are observed only in the SEM images of the surface 202 or 402 while substantially absent or visually unobservable in the surface 204 or 404.

In some examples, the surface features may include a series of pores and/or interstitial spaces adjacent to each other, such as the openings or spaces 604 formed on the surface. For example, the number of pores in the surface 202 or 402 may be greater in number than the surface 204 or 404.

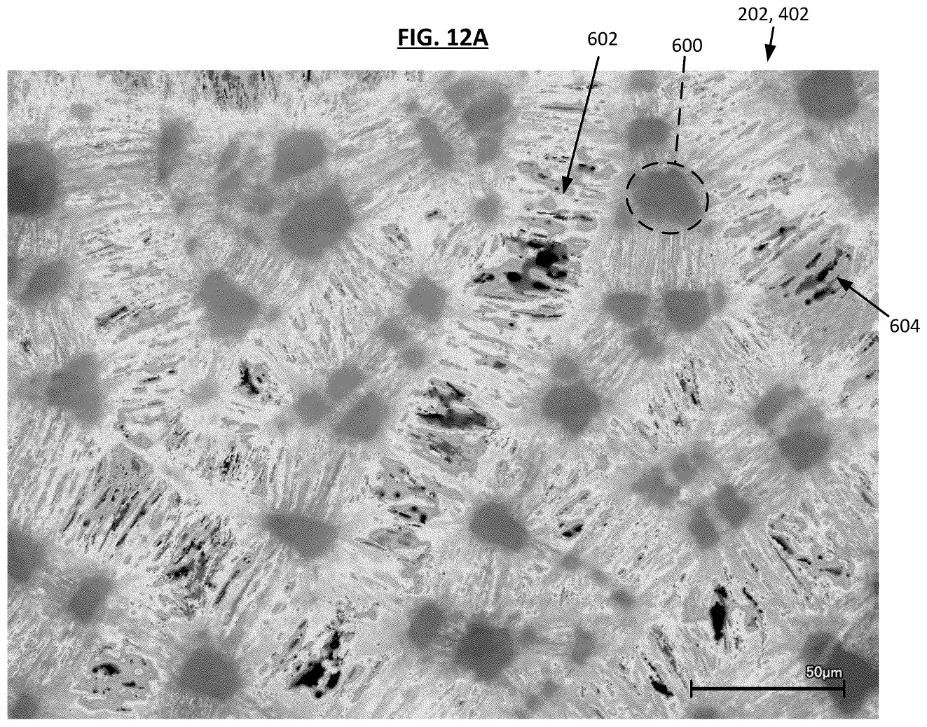

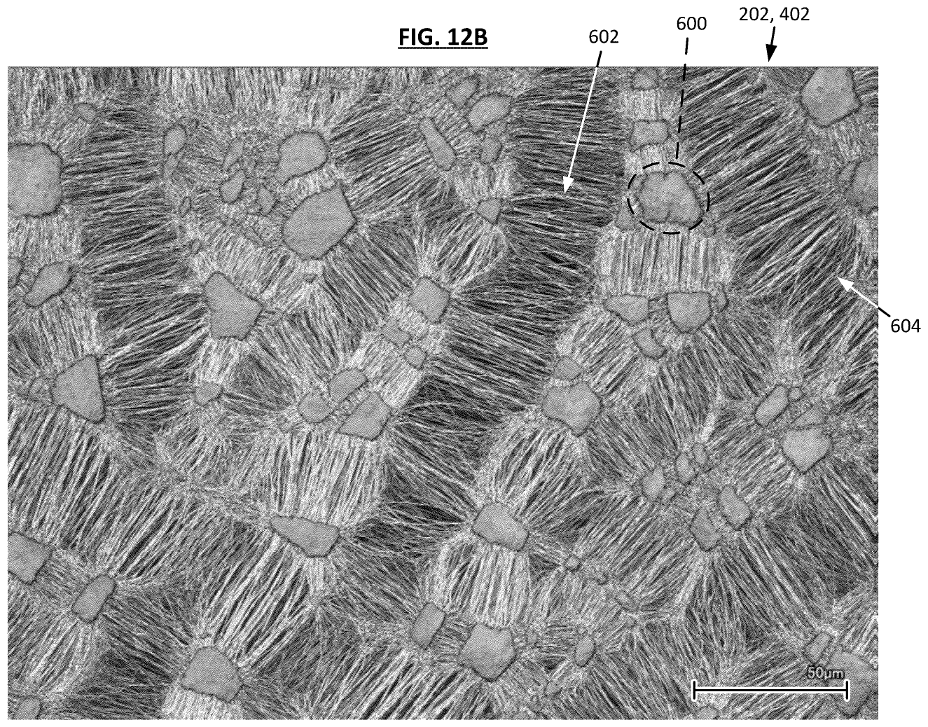

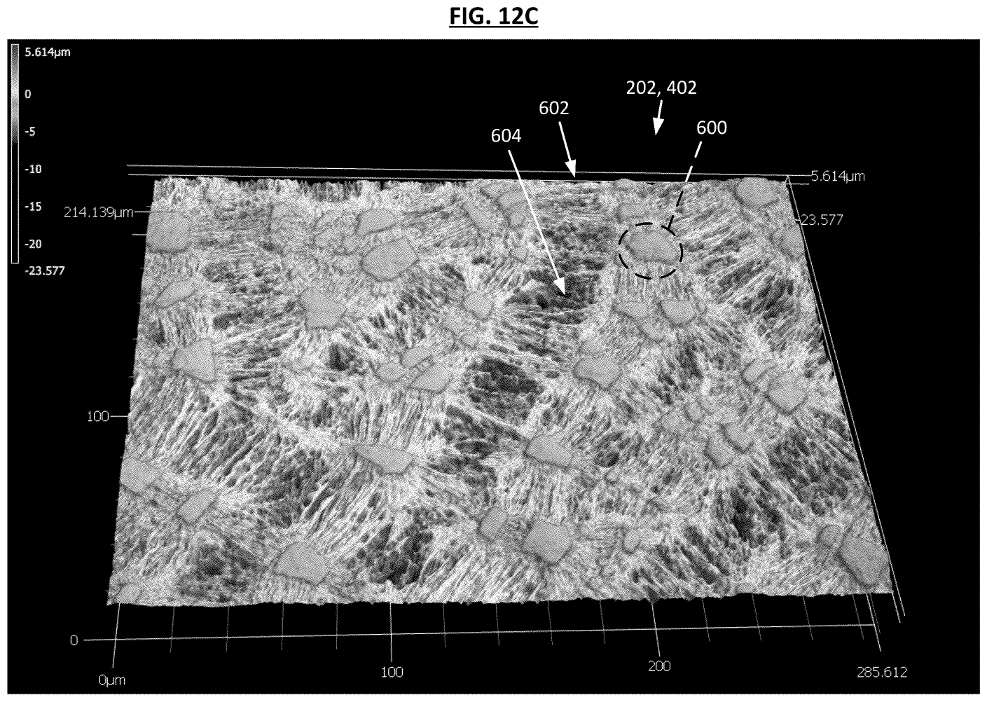

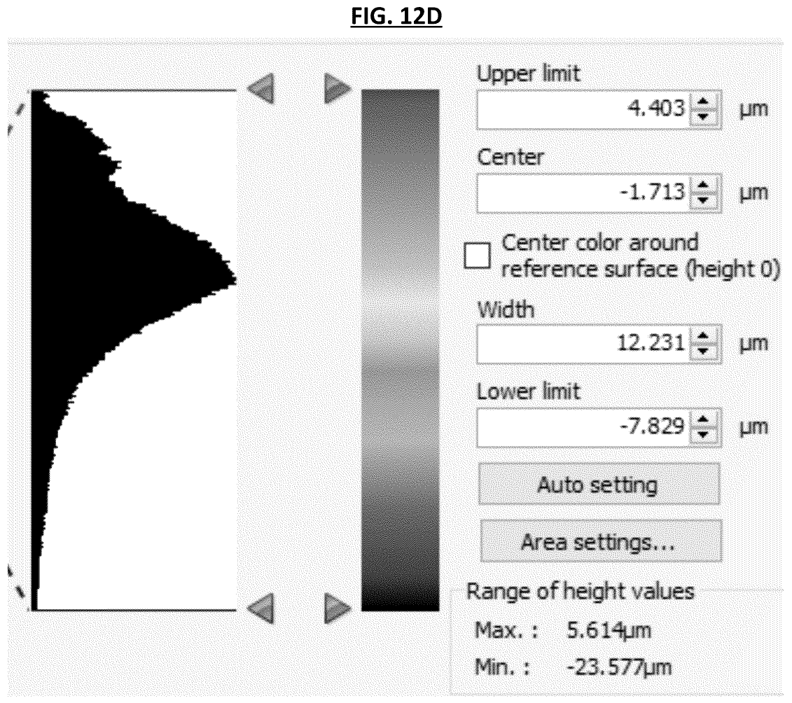

In some examples, the surface features may include roughness as defined by the texture and/or pore sizes on the surface. For example, a roughness (which may be an average roughness) of the surface 202 or 402 may be greater than a roughness of the surface 204 or 404. Definition and determination of surface roughness will be explained in greater detail in view of FIGS. 12A through 12D.

In some examples, the surface features may include a difference in depths or a maximum depth as measured with respect to the surface. For example, the surface 202 or 402 may define a greater depth into the body portion from the surface than does the surface 204 or 404. For example, the thickness or depth of the surface 204, 404 may be approximately 10% or less, 20% or less, 30% or less, 40% or less, 50% or less, or any other suitable value or range therebetween with respect to the thickness or depth of the surface 202, 402. The difference in thicknesses or depths may facilitate tissue ingrowth deep into the material and may allow the material to have a relatively thin layer to arrest further tissue ingrowth into the inner surface. Definition and determination of surface depths will be explained in greater detail in view of FIGS. 12A through 12D.

The body 200 may be formed of a compliant material having a thickness defining an ingrowth portion such as the first surface 402 that facilitates tissue ingrowth and a boundary portion such as the second surface 404 that prevents or inhibits tissue ingrowth. The ingrowth portion may correspond to the external surface 202, and the boundary portion may define the internal reservoir 206, which may have a fixed internal volume, within the body 200. The ingrowth portion may be characterized by visible microporous surface features at a first magnification, and the boundary portion may be characterized by an absence of visible microporous surface features at the first magnification. As described in association with other embodiments, the first magnification may be 50×, optionally 100×, optionally 500×, or optionally 1000×, or any range or value therebetween.

In some examples, the surface features may be defined by a plurality of fibrils extending between a plurality nodes that present a visually perceptible presence of the fibrils extending between the nodes in the external surface at a predetermined magnification, such as a microstructure defined by a plurality of fibrils extending between a plurality of nodes in which the microstructure is more visible at the external surface than the internal surface, or a fiber microstructure. For example, in the magnifications of FIGS. 9A and 9B as well as in FIGS. 10A and 10B, presence of the nodes 600 and the fibrils 602 extending therebetween (as well as the openings or spaces 604 defined by the fibrils 602) are visually perceptible or observable in the SEM images captured of the surface 202, 402 (FIGS. 9A and 10A). However, such nodes and fibrils are less observable, or in some examples unobservable, in the SEM images captured of the surface 204, 404 (FIGS. 9B and 10B) such that the two surfaces are distinguishable due to the differences in the surface features when each of the two surfaces is observed in the same magnification. Although the nodes and fibrils are observable in the 500× and 1000× magnifications in the aforementioned examples, it should be understood that, in some examples, the surface features may be observable in smaller magnifications, such as in 50× and 100× magnifications.

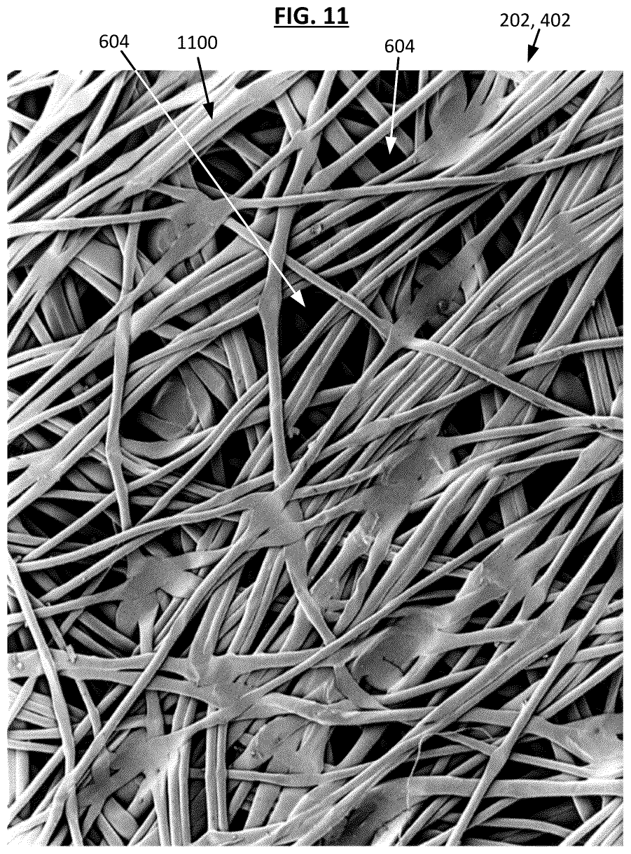

In some examples, the surface features may include a plurality of fibers 1100 forming a microstructure defining a structure of the body portion 200 as shown in FIG. 11. In some examples, the fibers may be nonwoven, e.g., spunbond, meltblown, direct spun, solution spun, gel spun, or electrospun materials. For example, the fibers may be spunbond polymer(s) including but not limited to spunbond PTFE, spunbond polypropylene, etc. As defined herein, fibers differ from fibrils in that the fibrils are generally disposed between two or more nodes that are formed in the body portion 200, whereas fibers may form a structure/microstructure such as a fiber matrix, a fiber web, a fiber network, or an interconnected fiber microstructure, for example. As shown in FIG. 11, the fibers 1100 may be interconnected, intertwined, or interwoven such that the fibers may be attached or bonded to other fibers where the attachment and/or interwovenness facilitate providing structural support for the fibers. In some examples, the fibers 1100 are deposited over each other and subsequently pressed together upon heating a portion of the fibers to a melting point such that at least a portion of the fibers are melted or deformed to at least partially bond to each other as shown. In some examples, the fibers 1100 may define therebetween a plurality of openings or spaces 604 which may be interstitial spaces within the structure which may have sufficient size to allow tissue ingrowth through the surface 202, 402.

In some examples, the surface feature is less present or minimally present at the surface 204 or 404 than at the other surface 202 or 402 such that the surface feature of the surface 204 or 404 is less visually observable or perceptible at the aforementioned magnification as compared to the other surface 202 or 402. In some examples, the feature may be not observable at all or entirely absent (e.g., the features are not able to be reliably discerned visually) at the surface 204 or 404 at the aforementioned magnification. As a non-limiting, illustrative example, FIGS. 9B and 10B showing the surface 204, 404 may be absent of the fibrils 602 connecting individual nodes 600 which are present or observable in FIGS. 9A and 10A on the surface 202, 402. As explained herein, FIGS. 9A and 9B show 500× magnification, and FIGS. 10A and 10B show 1000× magnification.

Referring to the external surface 202 or the first surface 402, the first porosity may be defined by a first average pore size. Such surface may be referred to as a tissue engagement surface. In some examples, the tissue engagement surface has a porosity extending into the engagement surface at an engagement depth to which an external tissue engages, in order to secure or anchor the body portion 200 or 400 at a suprachoroidal location in an eye at which the device 100 is implanted. In some examples, the engaging of the tissue engagement surface with the external tissue is observable after 10 days, 30 days, 50 days, 100 days, or longer. In some examples, sufficient tissue engagement may be defined by having enough cells (or sufficient number of cells) being integrated to affix or secure the implanted device at its location, or to minimize movement of the implanted device from its implanted location. In some examples, the engaging of the tissue engagement surface with the external tissue prevents or inhibits migration of the device 100 from the suprachoroidal location. In some examples, the ingrowth of the external tissue on the tissue engagement surface does not significantly inhibit fluid flow through the body portion 200 or 400. Referring to the internal surface 204 or the second surface 404, the second porosity may be defined by a second average pore size that is smaller than the first average pore size.

FIGS. 12A through 12C show different surface profilometry images of the external surface 202 or the first surface 402. In FIGS. 12A and 12B, displayed at the bottom corner is a line that represents 50 μm in each of the figures. In the color-coded surface profilometry image of FIG. 12A, the nodes 600 are in red, showing the highest elevation, and the fibrils 602 are in yellow or green, showing a lower elevation than the nodes 600, whereas the spaces or openings 604 are in blue or black, showing the lowest elevation (depression). As shown in the legend of FIG. 12C on the upper left-hand corner, the red portions represent the highest elevation of 5.614 μm whereas the black portions represent the lowest elevation of below about-6 μm, while the yellow portions represent the elevation of about 0 μm. As shown in the grid scale in the image, the distance between two consecutive lines on the grid of FIG. 12C is 20 μm. It should be understood that the values of these depths and elevations may vary according to how the profilometer is calibrated. For example, in the figures as shown, the profilometer is calibrated such that the 0 μm elevation corresponds substantially to the fibrils 602, as shown by the yellow portions of FIGS. 12A and 12C, such that the portions that are lower in elevation than the fibrils 602 are defined as the spaces or openings 604, and the portions that are higher in elevation than the fibrils 602 are defined as the nodes 600. In FIG. 12B, the monochromatic image shows the “cliffs” or edges of the nodes 600 as the elevation reduces from the highest points (such as the top surface of the nodes 600) to lower points (such as the fibrils 602), and the darkest portions indicate the spaces or openings 604 defined between the fibrils.

FIG. 12D shows a pop-up window which can be generated using any suitable surface profilometry software capable of calculating the features and properties of the surface based on the profilometry images as generated. In the examples shown, the images are generated using a 3D Optical Profiler (e.g., VK-X3000 series) or a 3D Optical Profilometer (e.g., VR-6000 series) as developed by Keyence Corporation (Osaka, Japan), although any other suitable optical profilometer may be used. According to the analysis performed by the software, the external surface 202 or the first surface 402 is shown to have a maximum height (or elevation) of 5.614 μm and a minimum height (or depth) of −23.577 μm, resulting in a difference of 29.191 μm, or about 30 μm.

The roughness of the surface, therefore, may be defined by the difference between the maximum height and the minimum height as determined using such surface profilometry, in which case the greater the difference, the greater the roughness. For example, the surface roughness of the surface 202, 402 may be defined as having a maximum-to-minimum height difference of at least 10 μm, at least 15 μm, at least 20 μm, at least 25 μm, at least 30 μm, at least 35 μm, at least 40 μm, or any other suitable value therein or range therebetween. In comparison, the surface roughness of the surface 204, 404 may be defined as having a maximum-to-minimum height difference of no greater than 10 μm, no greater than 7 μm, no greater than 5 μm, no greater than 4 μm, no greater than 3 μm, no greater than 2 μm, no greater than 1 μm, or any other suitable value therein or range therebetween. In some examples, the total thickness (that is, the distance measured between the maximum height of the external surface 202 or first surface 402 and the maximum height of the internal surface 204 or second surface 404, as appropriate) of the body portion 200 or 400 may be about 100 μm, about 120 μm, about 150 μm, about 170 μm, about 200 μm, or any other suitable value therein or range therebetween. In some examples, the maximum-to-minimum height difference may be defined as a percentage of the total thickness of the body portion 200, 400. For example, the surface 202, 402 may have a height difference of at least 5%, at least 10%, at least 15%, at least 20%, at least 25%, at least 30%, at least 35%, at least 40%, or any other suitable value therein or range therebetween, with respect to the total thickness of the body portion 200, 400. In comparison, the surface 204, 404 may have a height difference of no greater than 10%, no greater than 7%, no greater than 5%, no greater than 4%, no greater than 3%, no greater than 2%, no greater than 1%, or any other suitable value therein or range therebetween, with respect to the total thickness of the body portion 200, 400. The surface roughness of the external surface 202 or first surface 402 is greater than the surface roughness of the internal surface 204 or second surface 404.

Also, the depth of a surface may be defined as the maximum depth (or the minimum height, as measured by in FIG. 12D) of the surface. The surface 202, 402 may have a maximum depth or minimum height as measured with respect to the fibrils 602 as shown in FIGS. 12A and 12C, where the fibrils 602 generally occupy the center of the spectrum and act as the reference point for the other features such as the nodes 600 and the spaces or openings 604. Therefore, the maximum depth of the surface 202, 402 may be at least 10 μm, at least 15 μm, at least 20 μm, at least 25 μm, at least 30 μm, at least 35 μm, at least 40 μm, or any other suitable value therein or range therebetween. In comparison, the maximum depth of the surface 204, 404 may be no greater than 10 μm, no greater than 7 μm, no greater than 5 μm, no greater than 4 μm, no greater than 3 μm, no greater than 2 μm, no greater than 1 μm, or any other suitable value therein or range therebetween. Alternatively, the maximum depth of the surface 202, 402 may be at least 10%, at least 15%, at least 20%, at least 25%, at least 30%, at least 35%, at least 40%, or any other suitable value therein or range therebetween, with respect to the total thickness of the body portion 200, 400. In comparison, the maximum depth of the surface 204, 404 may be no greater than 10%, no greater than 7%, no greater than 5%, no greater than 4%, no greater than 3%, no greater than 2%, no greater than 1%, or any other suitable value therein or range therebetween, with respect to the total thickness of the body portion 200, 400. The maximum depth of the external surface 202 or first surface 402 is greater than the maximum depth of the internal surface 204 or second surface 404.

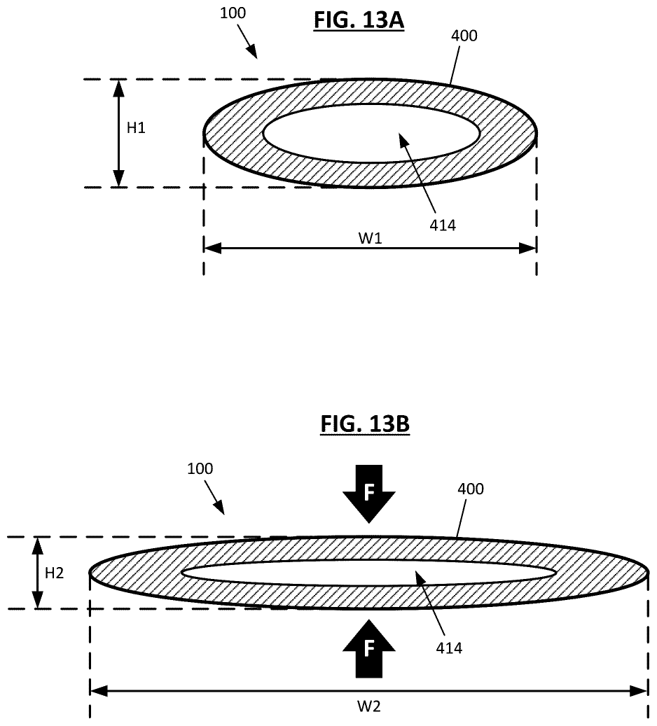

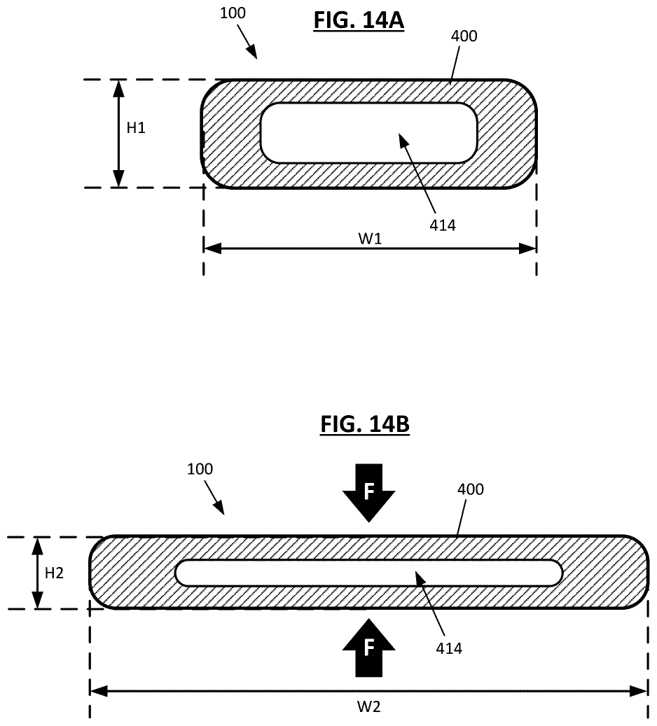

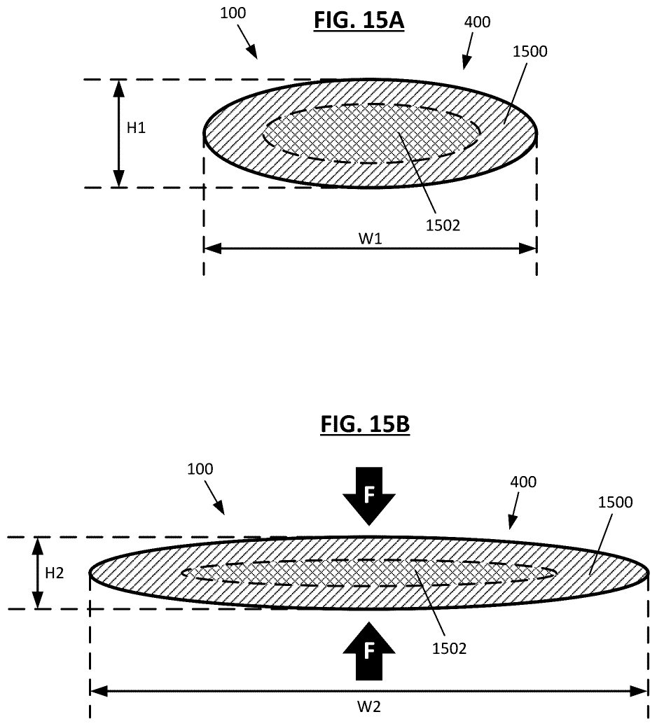

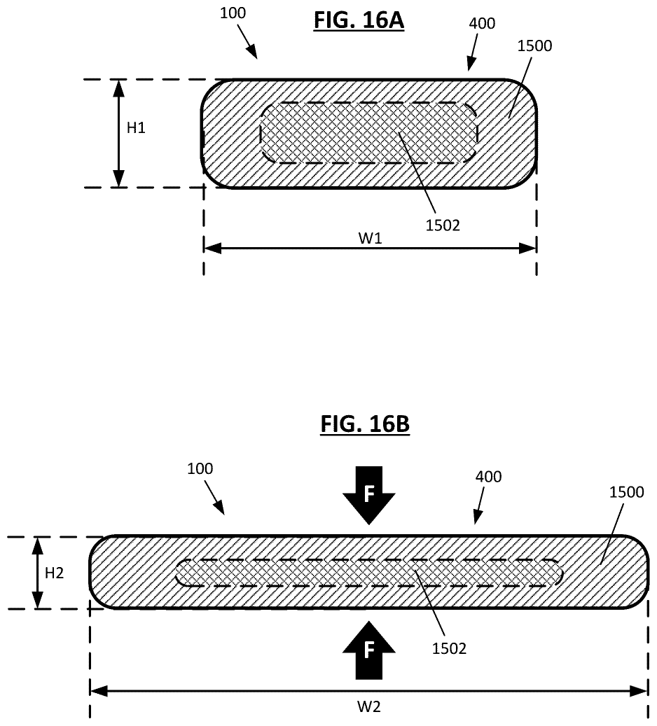

FIGS. 13A and 13B, FIGS. 14A and 14B, FIGS. 15A and 15B, and FIGS. 16A and 16B show examples of the device 100 and how the dimensions of the device 100 may change or transition from a first configuration to a second configuration in response to one or more directional forces (such as those as shown in black arrow with white “F” therein) according to different embodiments disclosed herein. The directional forces or external forces may be any forces that are applied to the device 100 from the surrounding environment in which the device 100 may be disposed, such as the forces from the tissue surrounding the device 100 after being implanted. Being able to flexibly respond to the forces being applied to the device 100 allows the device 100 to accommodate different environmental situations after being implanted, for example to improve comfort for the user. For example, FIGS. 13A, 13B, 15A, and 15B show devices 100 with a substantially ovular cross-section, and FIGS. 14A, 14B, 16A, and 16B show devices with a rounded rectangular cross-section.

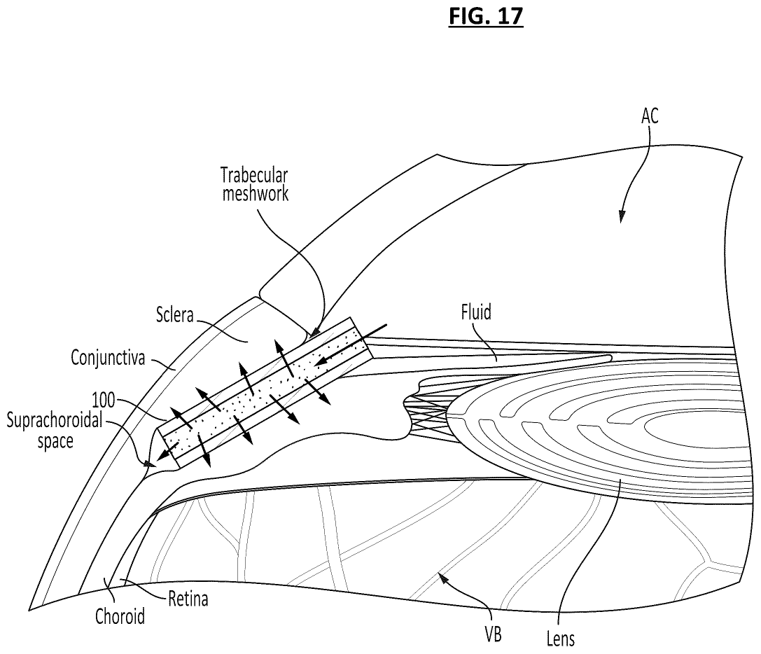

In FIGS. 13A and 13B as well as FIGS. 14A and 14B, the body portion 400 includes the internal space 414 into which fluid may flow from the AC as explained above, as shown in the arrows of FIG. 17, in which the fluid flows from the AC, through the device 100, and exits the device 100 at various locations toward the external surrounding, such as within the suprachoroidal space. In some examples, the body portion 400 may be open-ended on both ends of the device 100. In the absence of external forces, the body portion 400 assumes a first configuration that has a first height H1 and a first width W1, as shown in FIGS. 13A and 14A. The height may be less than the width, thereby forming a relatively flat cross-section. When external forces are applied, such as the forces as shown in arrows in FIGS. 13B and 14B, the body portion 400 assumes a second configuration that has a second height H2 and a second width W2, where H2<H1 and W2>W1.

In FIGS. 15A and 15B as well as FIGS. 16A and 16B, the body portion 400 includes a first portion 1500 and a second portion 1502 into which fluid may flow from the AC, as shown in the arrows of FIG. 17. The second portion 1502 (which may be referred to as an internal portion) is located more internally in the body portion 400 than the first portion 1500 (which may be referred to as an outer portion). In the absence of external forces, the body portion 400 has a first height H1 and a first width W1, as shown in FIGS. 15A and 16A. When external forces are applied, such as the forces as shown in arrows in FIGS. 15B and 16B, the body portion 400 assumes a second height H2 and a second width W2, where the second height is less than the first height, and the second width is greater than the first width, such that H2<H1 and W2>W1. In FIGS. 13A-13B, 14A-14B, 15A-15B, and 16A-16B, the body portion 400 may reversibly transition (e.g., transition back and forth while experiencing minimal or no permanent or irreversible defect) between the first configuration (with H1 and W1) and the second configuration (with H2 and W2) in the presence or absence of the external forces (F) being applied to the body portion 400 from the surroundings, such as the tissue surrounding the device 100 at the implantation site. The transitioning may allow the device 100 to fill extra space surrounding the device 100 at the implantation site, for example in the suprachoroidal space. Transitioning from the first configuration to the second configuration may cause the body 400 to exert a widening force against the surroundings to increase the width.

In some examples, the first and second portions 1500 and 1502 have different porosities with respect to each other. For example, the second portion 1502 may be more porous than the first portion 1500 to allow fluid to more readily enter the body portion 400 through the second (or internal) portion 1502. In some examples, the first and second portions 1500 and 1502 are made of different materials or different components that are combined together. In some examples, the first and second portions 1500 and 1502 are made of a single or unitary piece of material that is treated to form regions of different porosities. In some examples, the first and second portions 1500 and 1502 may include a transitional portion therebetween, such that the porosity changes gradually between the first and second portions 1500 and 1502, in which case the transitional portion may have one or more porosities that are greater than a porosity of the first portion 1500 and less than a porosity of the second portion 1502. Porosity may be measured using any suitable measurement, including but not limited to calculating average pore sizes, measuring the time it takes for a predetermined amount of fluid (such as water) to pass through the portions, and/or measuring the time it takes for a predetermined amount of gas (such as air) to pass through the portions at a predetermined pressure, for example.

The aforementioned configurations differ from the examples shown in lanchulev T, et al., such as CyPass®, iStent Supra®, MINIject®, and BioStent®. For example, CyPass® and iStent Supra® are described as rigid and non-conforming, whereas BioStent® is made using biotissue such as the decellularised scleral allograft tissue, which may lose the effect of maintaining a drainage pathway for aqueous humor to flow from the anterior chamber of the eye over time as the biotissue is absorbed into the tissue surrounding the device. The MINIject®, on the other hand, is manufactured such that the sizes of the connections between pores are controlled with high uniformity throughout the entire volume of the device, thereby having a uniform internal pore size of 27 μm, as explained in Grierson et al. (2020). “A novel suprachoroidal microinvasive glaucoma implant: in vivo biocompatibility and biointegration.” BMC Biomedical Engineering. (2020) 2:10. doi: 10.1186/s42490-020-00045-1. PMID: 33073174; PMCID: PMC7556975. Beneficially, in some examples, having variable pore sizes or porosities throughout the device 100 may beneficial in providing a plurality of different drainage pathways for the fluid to pass through the device 100 as explained above, in order to better reduce the IOP.

Various methods of controlling fluid pressure within an eye may be contemplated using the suprachoroidal implantable device 100 as disclosed herein. For example, the device 100 may be provided by the practitioner or surgeon and the disposed in a subconjunctival location of the eye as shown in any one of FIG. 1, 2C, 3C through 3E, 4D, 5D, or 17 as suitable according to the configuration of the device 100 that is being used for the treatment. Fluid pressure in the eye is controlled using the device 100 by directing a fluid through the compliant material of the device 100. Also disclosed herein are various methods of reducing a fluid pressure of a fluid within an eye. For example, the device 100 may be disposed in the subconjunctival location of the eye as shown in any one of FIG. 1, 2C, 3C through 3E, 4D, 5D, or 17 as suitable according to the configuration of the device 100 that is being used for the treatment. The fluid pressure is reduced by conveying the fluid though the compliant material of the device 100 from a high-pressure location of the eye to a low-pressure location of the eye. The high-pressure location may be located in the AC, and the low-pressure location may be located in the subconjunctival location or a surrounding space or region proximal to the location in which the device 100 is disposed. Tissue ingrowth may follow after the implanting procedure is finished, such that tissue may be allowed to grow partially into the device 100 after implant over a period of time. Generally, the first porosity facilitates tissue ingrowth at the external surface 202 or first surface 402 and the second porosity prevents, or substantially inhibits, tissue ingrowth through the internal surface 204 or second surface 404, as explained above.

In view of FIGS. 2A through 2C, in some examples, the device 100 may have a fixed volume, while in some examples, the device 100 has an adjustable internal volume, such that the practitioner or surgeon may dispose the device 100 in the subconjunctival location of the eye and subsequently adjust in situ the internal volume of the internal reservoir 206 in response to disposing the device 100 in the subconjunctival location of the eye. The adjustment may be performed using a needle of a syringe, for example. Other mechanisms for internal volume adjustment (e.g., shape memory materials, chemical or electrochemical reactions, or other mechanisms) are also contemplated.

In some examples, the sealable conduit 300 described in association with FIGS. 3A through 3E may be provided by the practitioner or surgeon, such that the second end 304 of the conduit 300 is fluidly coupled with the reservoir 206, while the first end 302 is disposed between a conjunctival tissue and a scleral tissue of the eye or in an AC of the eye, for example. In some examples, the practitioner or surgeon seals the first end 302 of the conduit 300 to inhibit fluid communication therethrough. The sealing of the first end 302 may be performed after the device 100 is implanted in the intended location in the eye, as explained above. In some examples, if the first end 302 is disposed in the AC of the eye, the practitioner or surgeon may fluidly couple the AC and the first end 302 of the conduit 300 to facilitate fluid communication therethrough from the internal surface 204 to the external surface 202 in response to a fluid pressure applied from the AC.

The device 100 described in association with FIGS. 4A through 4F as well as FIGS. 5A through 5D may be provided by the practitioner or surgeon, and the device 100 may be disposed in a subconjunctival location of the eye such that the open end 408 of the device 100 is fluidly coupled with the AC of the eye. After the implant, the first surface 402 may facilitate tissue ingrowth therein, and the second surface 404 may prevent or inhibit tissue ingrowth therethrough. The tissue ingrowth, as described above, may take any suitable number of days after the implant.

Various methods of manufacturing the suprachoroidal implantable device 100 may be contemplated as disclosed herein. For example, a suitable compliant material is provided, and the body portion 200 or 400 of the body portion is formed using the compliant material, such that the body portion 200 or 400 has the external surface 202 or first surface 402 as well as the internal surface 204 or second surface 404 as described above.

In some examples, such as the devices 100 shown in FIGS. 2A and 2B, the body portion 200 is formed using the compliant material, and the body portion 200 is provided with a filler material 208 therein to define the internal reservoir 206 with a fixed volume. In some examples, the body portion 200 is formed using the compliant material such that the internal reservoir 206 has an internal volume that is adjustable in situ. In such examples, such as the devices 100 shown in FIGS. 3A and 3B, the sealable conduit 300 may be provided such that the second end 304 is fluidly coupled with the internal reservoir 206 to facilitate such in situ adjustment to the internal volume of the internal reservoir 206.

In some examples, such as the devices 100 shown in FIGS. 4A through 4C, 4E, and 5A through 5C, the manufacturing method or process may include wrapping a compliant material around a mandrel, followed by applying heat treatment to the compliant material to form the body portion 400 having a closed end 406 and an open end 408 which, when implanted, would be fluidly coupled with the AC of an eye. After the heat treatment, the body portion 400 would have the first surface 402 and the second surface 404 according to any one or more embodiments as explained above, where the first surface 402 facilitates tissue ingrowth when the device 100 is implanted. For example, the first surface 402 may be less uniform than the second surface 404 at a certain magnification. In some examples, the body portion 400 may have a variable porosity that transitions from a first porosity located proximate the first surface 402 to a second porosity less than the first porosity and located proximate the second surface 404. In some examples, the first surface 402 may have the first porosity to facilitate tissue ingrowth into/through the first surface 402 to a desired depth, and the second surface 404 may have the second porosity to prevent, or substantially inhibit tissue ingrowth therethrough.

In some examples, referring to FIG. 4F, the manufacturing method or process may also include, prior to wrapping a compliant material (or a primary compliant material) around the mandrel, wrapping a secondary compliant material around the mandrel, such that the primary compliant material is wrapped around the secondary compliant material. The heat treatment may be applied to both of the compliant materials, such that the resulting body portion 400 includes both the external layer 410 (formed by the primary compliant material) and the internal elastic support structure 412 (formed by the secondary compliant material). Specifically, applying heat treatment to the compliant material forms the external microporous layer 410 which defines both the first surface 402 and the second surface 404 of the body portion 400, and applying heat treatment to the secondary compliant material forms the internal elastic support structure 412 disposed within the external microporous layer 410. The internal elastic support structure 412 defines an internal space of the device 100 such that the internal elastic support structure 412 has a third porosity greater than the first porosity and the second porosity to facilitate fluid communication between the internal volume or space 414 and the second surface 404, as explained above.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. A suprachoroidal implantable device comprising:
a body portion formed of a compliant material having an external surface and an internal surface defining an internal reservoir of the body portion with a fixed volume, the body portion having a variable porosity that transitions from a first porosity located proximate the external surface to a second porosity less than the first porosity and located proximate the internal surface,
wherein the first porosity facilitates tissue ingrowth at the external surface and the second porosity inhibits tissue ingrowth through the internal surface.

2. The device of claim 1, wherein the first porosity is defined by a first average pore size, and the second porosity is defined by a second average pore size that is smaller than the first average pore size.

3. The device of claim 1, wherein the external surface is a tissue engagement surface, the tissue engagement surface has a porosity extending into the engagement surface at an engagement depth to which an external tissue engages in order to secure or anchor the body portion at a suprachoroidal location in an eye at which the device is implanted, and the porosity is selected such that the external tissue is observable to engage the engagement surface at the engagement depth after 30 days.

4. The device of claim 1, wherein the internal reservoir includes a filler material encapsulated therein, and the filler material includes a medicament selected to pass through the compliant material from the internal surface to the external surface over a period of 30 days.

5. A suprachoroidal implantable device comprising:
a body portion formed of a compliant material having an external surface and an internal surface defining an internal reservoir of the body portion with a fixed volume, the external surface having a first porosity and the internal surface having a second porosity less than the first porosity,
wherein the first porosity facilitates tissue ingrowth at the external surface and the second porosity inhibits tissue ingrowth through the internal surface.

6. The device of claim 5, wherein the first porosity is defined by a first average pore size, and the second porosity is defined by a second average pore size that is smaller than the first average pore size.

7. The device of claim 5, wherein the external surface is a tissue engagement surface, the tissue engagement surface has a porosity extending into the engagement surface at an engagement depth to which an external tissue engages in order to secure or anchor the body portion at a suprachoroidal location in an eye at which the device is implanted, and the porosity is selected such that the external tissue is observable to engage the engagement surface at the engagement depth after 30 days.

8. The device of claim 5, wherein the internal reservoir includes a filler material encapsulated therein, and the filler material includes a medicament selected to pass through the compliant material from the internal surface to the external surface over a period of 30 days.

9. A suprachoroidal implantable device comprising:
a body portion formed of a compliant material having an external surface and an internal surface defining an internal reservoir of the body portion with an internal volume that is adjustable in situ, the body portion having a variable porosity that transitions from a first porosity located proximate the external surface to a second porosity less than the first porosity and located proximate the internal surface,
wherein the first porosity facilitates tissue ingrowth at the external surface and the second porosity inhibits tissue ingrowth through the internal surface.

10. A suprachoroidal implantable device comprising:
a body portion formed of a compliant material having an external surface and an internal surface defining an internal reservoir of the body portion with an internal volume that is adjustable in situ, the external surface having a first porosity and the internal surface having a second porosity less than the first porosity,
wherein the first porosity facilitates tissue ingrowth at the external surface and the second porosity inhibits tissue ingrowth through the internal surface.

11. A suprachoroidal implantable device comprising:
a body portion having a closed end and an open end fluidly couplable with an anterior chamber (AC) of an eye,
the body portion comprising a first surface and a second surface, the body portion having a variable porosity that transitions from a first porosity located proximate the first surface to a second porosity less than the first porosity and located proximate the second surface,
wherein the first porosity facilitates tissue ingrowth at the first surface and the second porosity inhibits tissue ingrowth through the second surface.

12. The device of claim 11, wherein the body portion is a tubular construct having a first end and a second end, wherein the first end is closed to form the closed end of the body portion and the second end is retained in an open configuration to form the open end of the body portion.

13. The device of claim 11, wherein the body portion has a substantially circular cross-section.

14. The device of claim 11, wherein the body portion has a substantially ovular or rounded rectangular cross-section.

15. The device of claim 14, wherein, in an absence of external forces, the body portion assumes a first configuration that has a first height and a first width, and in response to the external forces being applied to the body portion, the body portion assumes a second configuration that has a second height and a second width, wherein the second height is less than the first height, and the second width is greater than the first width.

16. The device of claim 15, wherein the body portion reversibly transitions between the first configuration and the second configuration in the presence or absence of the external forces being applied to the body portion.

17. The device of claim 11, wherein the body portion includes:
an external microporous layer, and
an internal elastic support structure disposed within the external microporous layer and defining an internal space,
wherein the external microporous layer defines both the first surface and the second surface, and wherein the internal elastic support structure has a third porosity.

18. The device of claim 17, wherein the third porosity is greater than the first porosity and the second porosity to facilitate fluid communication between the internal space and the second surface.

19. A suprachoroidal implantable device comprising:
a body portion having a closed end and an open end fluidly couplable with an anterior chamber (AC) of an eye,
the body portion comprising a first surface and a second surface, the first surface having a first porosity, and the second surface having a second porosity less than the first porosity,
wherein the first porosity facilitates tissue ingrowth at the first surface and the second porosity inhibits tissue ingrowth through the second surface.

20. The device of claim 19, wherein the body portion is a tubular construct having a first end and a second end, wherein the first end is closed to form the closed end of the body portion and the second end is retained in an open configuration to form the open end of the body portion.

21. The device of claim 19, wherein the body portion has a substantially circular cross-section.

22. The device of claim 19, wherein the body portion has a substantially ovular or rounded rectangular cross-section.

23. The device of claim 22, wherein, in an absence of external forces, the body portion assumes a first configuration that has a first height and a first width, and in response to the external forces being applied to the body portion, the body portion assumes a second configuration that has a second height and a second width, wherein the second height is less than the first height, and the second width is greater than the first width.

24. The device of claim 23, wherein the body portion reversibly transitions between the first configuration and the second configuration in the presence or absence of the external forces being applied to the body portion.

25. The device of claim 19, wherein the body portion includes:
an external microporous layer, and
an internal elastic support structure disposed within the external microporous layer and defining an internal space,
wherein the external microporous layer defines both the first surface and the second surface, and wherein the internal elastic support structure has a third porosity.

26. The device of claim 25, wherein the third porosity is greater than the first porosity and the second porosity to facilitate fluid communication between the internal space and the second surface.
